(19) United States
(12) Patent Application Publication
(10) Pub. No.: US 2008/0317806 A1
(43) Pub. Date: Dec. 25, 2008

(54) Antiparasitic Compounds

(75) Inventors: Wayne Morris Best, Western Australia (AU); Colette Gloria Sims, Western Australia (AU); Richard Christopher Andrew Thompson, Western Australia (AU); Simon Andrew Reid, Western Australia (AU); Anthony Armson, Western Australia (AU); James Alexander Reynoldson, Western Australia (AU)

Correspondence Address: HOWREY LLP - DC, C/O IP DOCKETING DEPARTMENT, 2941 FAIRVIEW PARK DR, SUITE 200, FALLS CHURCH, VA, 22042-2924, US

(73) Assignee: Murdoch University, Murdoch, Western Australia (AU)

(21) Appl. No.: 11/911,099

(22) PCT Filed: Apr. 11, 2006
(86) PCT No.: PCT/AU06/00488

§ 371 Date: Aug. 7, 2008

(30) Foreign Application Priority Data
Apr. 11, 2005 (AU) 2005901779

(51) Int. Cl. A61F 2/04; C07D 241/04; A61K 31/495; C07D 265/30; A61K 31/5375; C07C 211/46; A61P 33/00; A61K 31/136; A61K 31/54; C07D 279/12; A61K 9/12

(52) U.S. Cl. 424/422; 544/382; 514/255.01; 514/238.8; 544/164; 564/441; 514/646; 514/227.5; 544/58.1; 424/45

(57) ABSTRACT

A compound of Formula (A), wherein R¹ is C₁-C₅ alkyl, C₃-C₆ branched alkyl, C₄-C₇ cycloalkyl, C₈-C₁₂ fused or bridged polycycloalkyl, or heterocyclic ring, where any of the preceding alkyl, cycloalkyl or heterocyclic ring groups may be singly or multiply substituted with X; R² is H or R¹; and X is halo, carbonyl carboxylic acid, carboxylic ester, carboxamide, substituted carboxamide, hydroxy, alkoxy, thioalkyl, sulphoxide, sulphone, sulphonamide, substituted sulphonamide, phenoxy, substituted phenoxy, phenyl, substituted phenyl, amino, substituted amino (including quaternary ammonium salts), N-oxide, imino, 5-7 membered heterocycle, or substituted heterocycle.

The present application is filed Pursuant to 35 U.S.C. 371 as a U.S. National Phase application of International Patent Application No. PCT/AU2006/000488 which was filed Apr. 11, 2006, claiming the benefit of priority of Australian Patent Application No. 2005901779, which was filed on Apr. 11, 2005.

### FIELD OF THE INVENTION

The present invention relates to anti-parasitic compounds and their use to treat parasite infections and more particularly to dinitroaniline compounds and their use as parasiticides.

### BACKGROUND ART

Diseases caused by parasite infections far outnumber diseases caused by other infectious agents. It is estimated that one billion people are infected with the helminthic parasite roundworm alone and that eight hundred million people are infected with hookworm. This does not include the billions of animals infected with parasites.

Parasitic infection vary from severe to very mild infections; and may sometimes be so mild that no clinical or subclinical infection can be detected by measuring weight loss, food consumption, blood parameters, or histopathology. The infectious parasites affect the host in many ways depending on the tissue tropism (site organ and tissue preference) of the specific parasite; the number of parasitic oocysts ingested by the subject in the initial infection; and the pathogenicity of the parasitic species. Often, minimal to no clinical evidence of infection is observed; but the loss to the subject is primarily as a depressed growth and impaired feed conversion. In animals destined for consumption by humans, the infection often results in a downgrading of quality at processing of the carcass, such that the animal no longer qualifies as food fit for human consumption.

Parasite infections in subjects, including humans, are typically treated by chemical drugs. However, the approach of repeated administration of drugs, both prophylactically and therapeutically, has resulted in the selection and survival of drug-resistant parasitic strains that no longer respond to treatment. Furthermore, many of the previously used and currently employed chemical drugs interfere with the host metabolism and are harmful to the subject being treated, often resulting in toxicity or decreased weight gains and feed efficiency when used at high doses. As larger doses become required due to the build up of resistance, the side effects become even greater. Additionally, the life cycle of most parasites includes a variety of life forms, each of which presents different targets, and challenges, for chemical therapy.

Parasitic infections of great economic significance both directly to humans and to the animals we breed for food, companionship, and other economic benefits include helminthic parasites, Giardia, Cryptosporidium, malarial species etc.

There has been a continuing effort to develop methods for treating infected animals and controlling the spread of helminthic parasite infections. Broad spectrum anthelmintic agents such as aminoglycoside antibodies, organophosphorous compounds, benzimidazoles, organic arsenic compounds, piparazines, imidoylureas are a few among many anthelmintic agents that have been used to treat and control the spread of helminthic parasite infections. These anthelmintic agents generally function by destroying helminthic parasites in various developmental stages including adults, larvae, and eggs. However, many of these compounds are toxic to the host in effective dosages, difficult to prepare or synthesize, expensive, or produce adverse side effects when administered to the host animal. Therefore, there exists a continuing need for new and more effective methods for controlling helminthic parasites.

Plasmodium, the agent responsible for malaria, is an obligate intracellular parasite. More than ten years ago an urgent need for drugs against malaria was identified. The antibiotics currently in use, including the tetracyclines and clindamycin, for the treatment and prophylaxis of malaria have little action on pre-erythrocytic stages and a slow action on blood stages, but are used for treatment of drug resistant strains because of their safety rather than their efficacy. Furthermore, the rapid spread of resistance to chloroquine has heightened the need for relatively low cost prophylactic and therapeutic anti-malarial drugs. These include compounds that reverse resistance to chloroquine, compounds that act rapidly to treat falciparum malaria and others that can be administered by methods other than injection (to avoid the use of contaminated needles).

Cryptosporidiosis infection varies with host immune competence from mild, self-limiting diarrhoea to life-threatening enteritis complicated by extraintestinal disease. There is no reliable therapy for cryptosporidiosis. The problems of developing in vitro and in vivo methods of screening drugs, such as limited availability and poor reproducibility, have contributed to this lack of effective treatment. However, the major hindrance has been a lack of understanding of the parasite, its virulence and its interactions with the host's immune system.

Insofar as is presently known, therefore, both conventionally known major approaches to treating parasitic infections continue to impose major drawbacks and difficulties regarding their use, efficacy, and concomitant undesirable consequences. For these reasons, new methods for prophylactically and/or therapeutically treating parasitic infections in subjects such as humans and animals intended for human consumption have long been sought without apparent success. Accordingly, the introduction of efficacious anti-parasitic approaches and methods which are relatively simple, rapid, and easy to employ with large numbers of animals would be recognized by practitioners skilled in this art as a major advance and improvement in this field.

Other objects, features, and advantages of the instant invention may be determined from the following description and examples.

### SUMMARY OF THE INVENTION

The present invention provides a compound of Formula A:

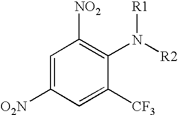

wherein R₁ is C₁-C₅ alkyl, C₃-C₆ branched alkyl, C₄-C₇ cycloalkyl, C₈-C₁₂ fused or bridged polycycloalkyl, or heterocyclic ring, where any of the preceding alkyl, cycloalkyl or heterocyclic ring groups may be singly or multiply substituted with X;

R₂ is H or R₁; and

X is halo, carbonyl, carboxylic acid, carboxylic ester, carboxamide, substituted carboxamide, hydroxy, alkoxy, thioalkyl, sulphoxide, sulphone, sulphonamide, substituted sulphonamide, phenoxy, substituted phenoxy, phenyl, substituted phenyl, amino, substituted amino (including quaternary ammonium salts), N-oxide, imino, 5-7 membered heterocycle, or substituted heterocycle; or a pharmaceutically acceptable salt thereof. Preferably, R1 and R2 are connected by a bond to form a heterocyclic ring.

The present invention also provides a method for preparing a compound of Formula A, the method comprising the step of reacting 2-chloro-3,5-dinitrobenzotrifluoride with the corresponding HN(R₁)R₂.

The compounds of the present invention may be formulated into compositions for administration. Thus, the present invention also provides a composition comprising a therapeutically-effective amount of a compound of Formula A and a pharmaceutically acceptable carrier or diluent.

The compounds of the present invention have broad antiparasitic activity, and thus are useful for treating or preventing parasitic infections. Thus, the present invention also provides a method of treating a parasitic infection in a subject comprising the step of administering to the subject an effective amount of a compound of Formula A.

The present invention further provides for the use of a compound as described herein or a pharmaceutically acceptable salt thereof in the manufacture of a medicament for the treatment or prophylaxis of parasite infection in a subject in need thereof.

### BRIEF DESCRIPTION OF THE DRAWINGS

FIG. 1 is a plot of an amplification curve for cell culture controls and a cryptosporidial infection in the presence of a novel drug compound;

FIGS. 2A to 2E are tables of the various substituents that can be utilised to form preferred compounds of the invention;

FIGS. 3A to 3C are tables of some preferred forms of the compounds of the invention;

FIGS. 4A and 4B are tables of the antiparasitic activity of particular compounds of the present invention against T. rhodesiense, T. brucei, T. cruzi, L. donovani, E. multi, and C. elegans (the activity of 100 mM compound against C. elegans is indicated by the following symbols: − no effect; + slight reduction in growth and/or motility; ++ significant reduction in growth and/or motility; +++ all worms dead);

FIGS. 5A and 5B are tables of the antiparasitic activity of particular compounds of the present invention against Cryptosporidium, Giardia, and P. falciparum, and also set out cytotoxicity data.

### DESCRIPTION OF THE INVENTION

### Compounds

The present invention provides a compound of Formula A:

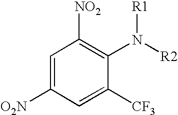

wherein R₁ is C₁-C₅ alkyl, C₃-C₆ branched alkyl, C₄-C₇ cycloalkyl, C₈-C₁₂ fused or bridged polycycloalkyl, or heterocyclic ring, where any of the preceding alkyl, cycloalkyl or heterocyclic ring groups may be singly or multiply substituted with X;

R₂ is H or R₁; and

X is halo, carbonyl, carboxylic acid, carboxylic ester, carboxamide, substituted carboxamide, hydroxy, alkoxy, thioalkyl, sulphoxide, sulphone, sulphonamide, substituted sulphonamide, phenoxy, substituted phenoxy, phenyl, substituted phenyl, amino, substituted amino (including quaternary ammonium salts), N-oxide, imino, 5-7 membered heterocycle, or substituted heterocycle.

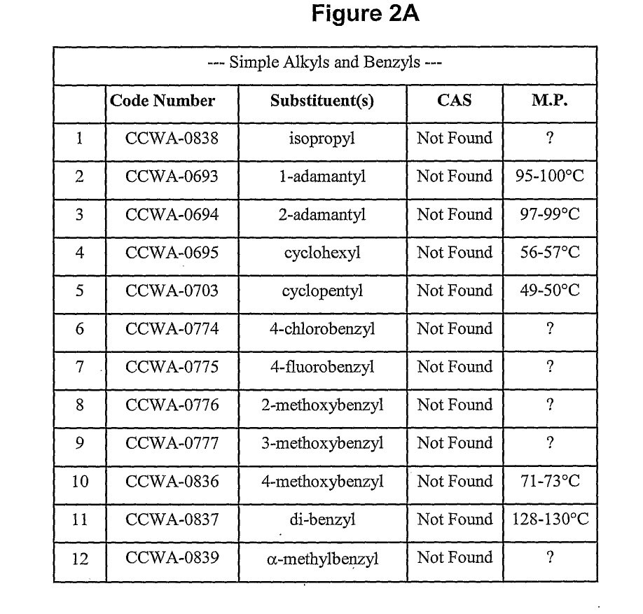

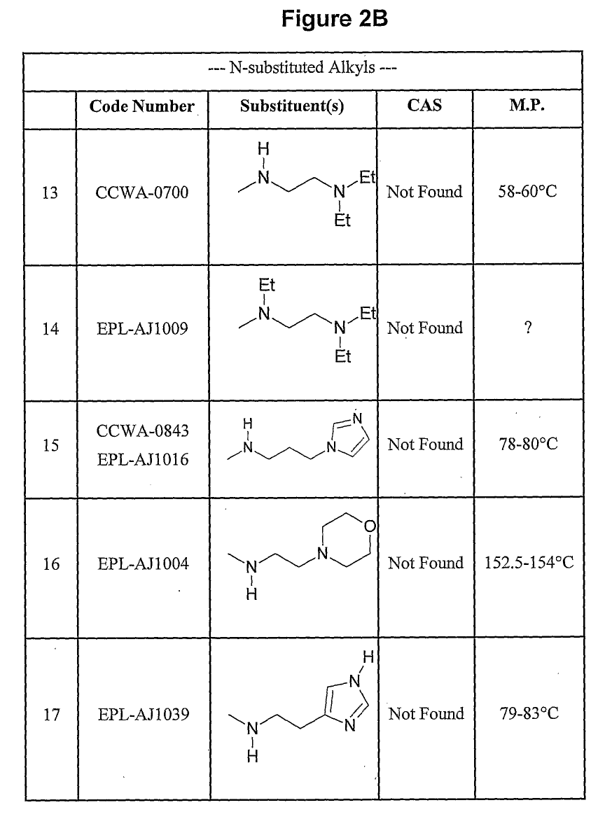

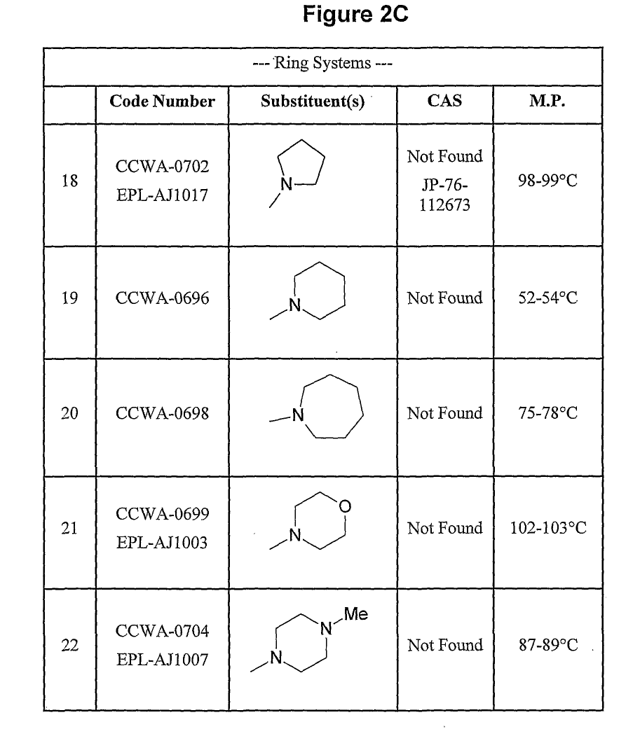

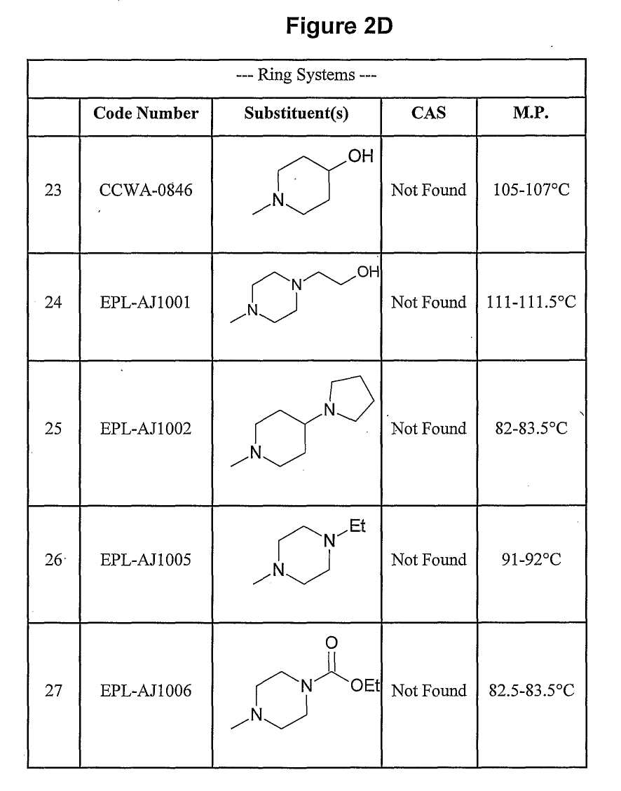

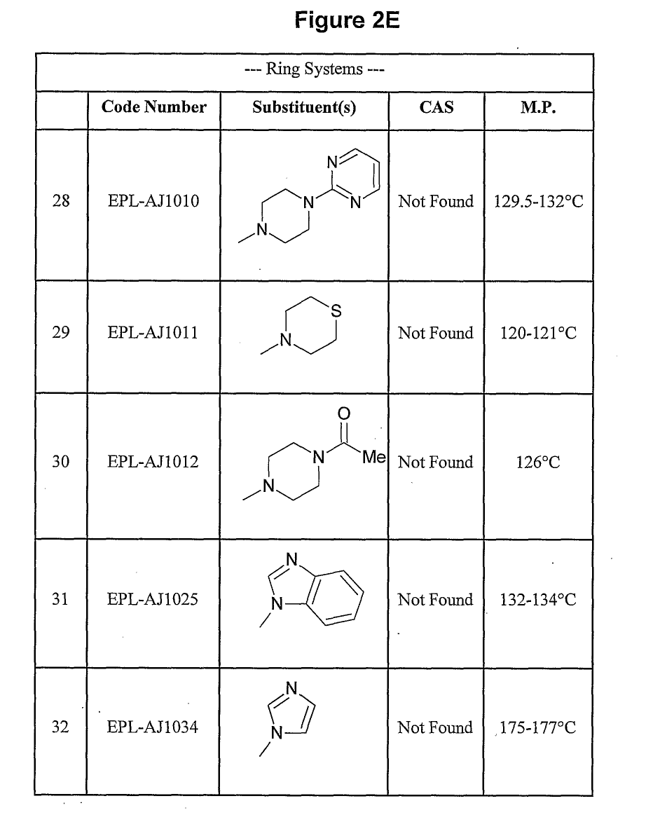

R₁ may be a substituent selected from the substituents listed in FIGS. 2A-2E.

R₁ and R₂ may be connected by a bond to form a heterocyclic ring. Preferably, the heterocyclic ring contains one or more heteroatoms.

In one form of the invention the compounds contain at least one ring system (in addition to 2,4-dinitro-6-(trifluoromethyl)analine ring) or at least one heteroatom in addition to the 2,4-dinitro-6-(trifluoromethyl)analine moiety. Preferably, N(R1)R2 is not pyrrolidino.

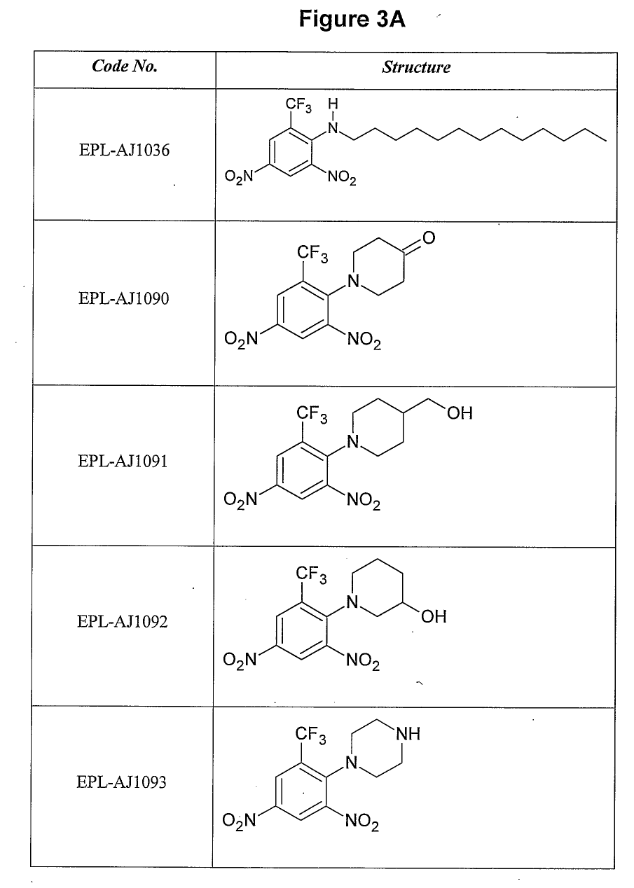

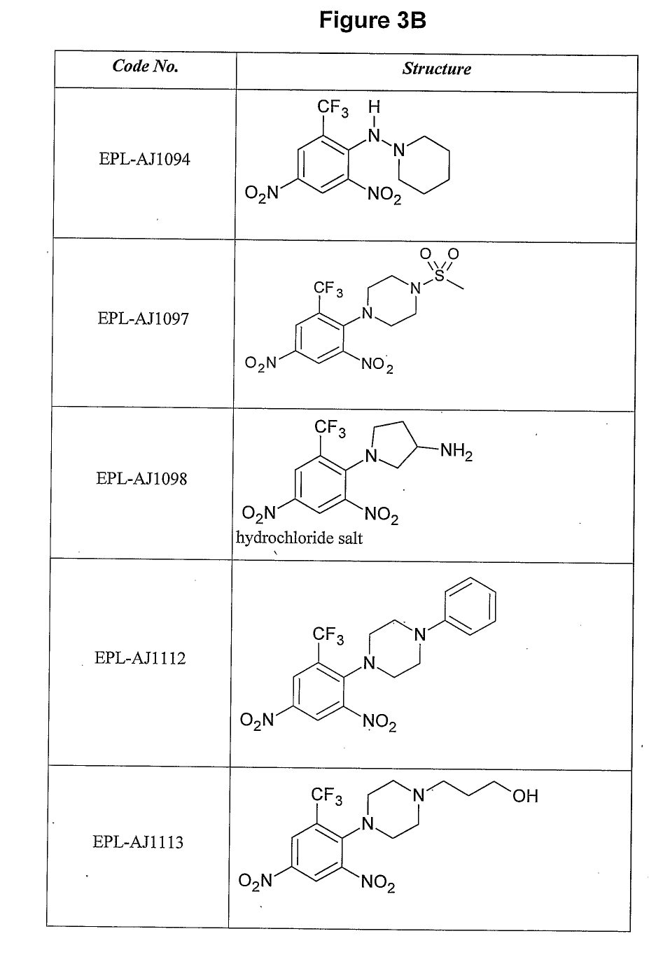

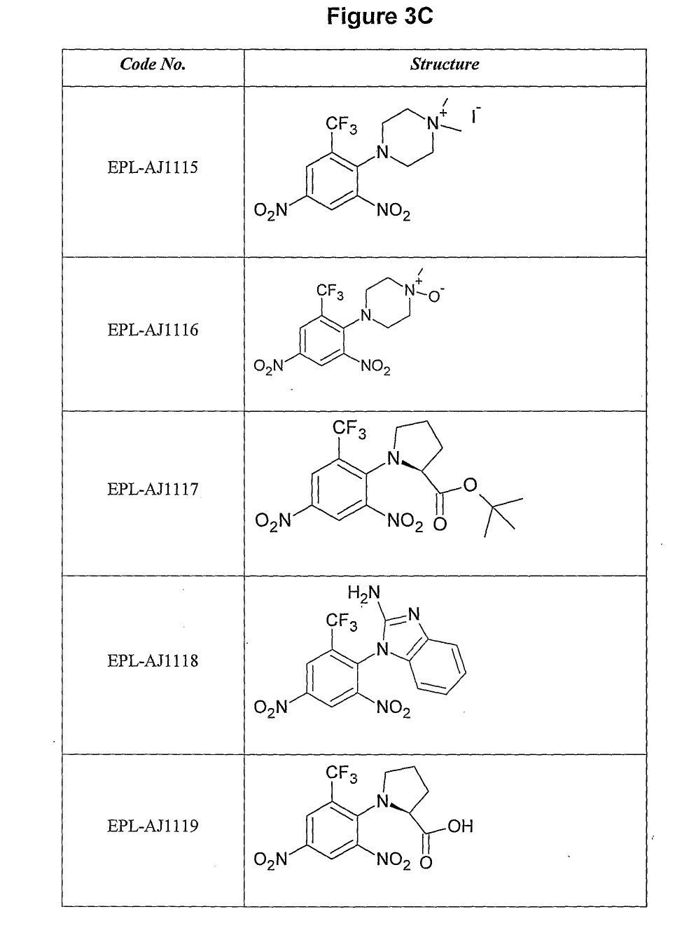

Preferably, the compound of the present invention is selected from the group comprising the compounds listed in FIGS. 3A to 3C

The compound of the present invention may be selected from the group consisting of: 1-(4-methyl-1-piperazinyl)-2,4-dinitro-6-(trifluoromethyl)benzene; 1-morpholino-2,4-dinitro-6-(trifluoromethyl)benzene; N-cyclopentyl-2,4-dinitro-6-(trifluoromethyl)aniline; and N-cyclopentyl-N-methyl-2,4-dinitro-6-(trifluoromethyl)aniline.

The compound of the present invention may be in the form of a pharmaceutically acceptable salt. Pharmaceutically acceptable salts for the purposes of the present invention include non-toxic acid addition salts formed with pharmaceutically acceptable acids. Examples include, but are not limited to, hydrochloride, hydrobromide, sulphate and phosphate, acetate, borate, nitrate, citrate, fumarate, gluconate, lactate, maleate, succinate and tartrate salts. Other salts include pharmaceutically acceptable metal salts such as non-toxic alkali metal salts, with bases. Examples include, but are not limited to sodium and potassium salts, ammonium and alkylammonium salts including tetralkylammonium salts.

### Methods of Preparing Compounds

The present invention also provides a method for preparing a compound of Formula A wherein R₁ is C₁-C₅ alkyl, C₃-C₆ branched alkyl, C₄-C₇ cycloalkyl, C₈-C₁₂ fused or bridged polycycloalkyl, or heterocyclic where any of the preceding alkyl, cycloalkyl or heterocyclic ring groups may be singly or multiply substituted with X; R₂ is H or R₁; and X is halo, carbonyl, carboxylic acid, carboxylic ester, carboxamide, substituted carboxamide, hydroxy, alkoxy, thioalkyl, sulphoxide, sulphone, sulphonamide, substituted sulphonamide, phenoxy, substituted phenoxy, phenyl, substituted phenyl, amino, substituted amino (including quaternary ammonium salts), N-oxide, imino, 5-7 membered heterocycle, or substituted heterocycle; the method comprising the step of reacting 2-chloro-3,5-dinitrobenzotrifluoride with the corresponding HN(R₁)R₂.

Preferably the reaction is carried out in the presence of a base or with excess of HN(R₁)R₂ if it is sufficiently basic.

### Compositions

The compounds of the present invention may be formulated into compositions for administration. Thus, the present invention also provides a composition comprising a therapeutically-effective amount of a compound of Formula A, wherein R₁ is C₁-C₅ alkyl, C₃-C₆ branched alkyl, C₄-C₇ cycloalkyl, C₈-C₁₂ fused or bridged polycycloalkyl, or heterocyclic ring, where any of the alkyl or cycloalkyl groups may be singly or multiply substituted with X; R₂ is H or R₁; R₁ and R₂ may be connected by a bond to form a heterocyclic ring; and X is halo, carbonyl, carboxylic acid, carboxylic ester, carboxamide, substituted carboxamide, hydroxy, alkoxy, thioalkyl, sulphoxide, sulphone, sulphonamide, substituted sulphonamide, phenoxy, substituted phenoxy, phenyl, substituted phenyl, amino, substituted amino (including quaternary ammonium salts), N-oxide, imino, 5-7 membered heterocycle, or substituted heterocycle; and a pharmaceutically acceptable carrier or diluent.

The precise composition of the present invention will vary according to a wide range of commercial and scientific criteria. Methods for the preparation of pharmaceutical compositions comprising one or more active ingredients are generally known in the art. Such compositions will generally be formulated for the mode of delivery that is to be used and will usually include one or more pharmaceutically acceptable carriers.

Generally, examples of suitable carriers, excipient and diluents include, without limitation, water, saline, ethanol, dextrose, glycerol, lactose, dextrose, sucrose sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, talc, magnesium stearate and mineral oil or combinations thereof. The formulations can additionally include lubricating agents, pH buffering agents, wetting agents, emulsifying and suspending agents, preserving agents, sweetening agents or flavouring agents.

### (a) Topicals

The pharmaceutical composition may be adapted for topical application. In this regard, various topical delivery systems may be appropriate for administering the compositions of the present invention depending upon the preferred treatment regimen. Topical formulations may be produced by dissolving or combining the compound of the present invention in an aqueous or nonaqueous carrier. In general, any liquid, cream, or gel, or similar substance that does not appreciably react with the compound or any other of the active ingredients that may be introduced into the composition and which is non-irritating is suitable. Appropriate non-sprayable viscous, semi-solid or solid forms can also be employed that include a carrier compatible with topical application and have a dynamic viscosity preferably greater than water.

Suitable formulations are well known to those skilled in the art and include, but are not limited to, solutions, suspensions, emulsions, creams, gels, ointments, powders, liniments, salves, aerosols, transdermal patches, etc, which are, if desired, sterilized or mixed with auxiliary agents, e.g., preservatives, stabilizers, emulsifiers, wetting agents, fragrances, colouring agents, odour controllers, thickeners such as natural gums etc. Particularly preferred topical formulations include ointments, creams or gels.

Ointments generally are prepared using either (1) an oleaginous base, i.e., one consisting of fixed oils or hydrocarbons, such as white petroleum or mineral oil, or (2) an absorbent base, i.e., one consisting of an anhydrous substance or substances which can absorb water, for example anhydrous lanolin. Customarily, following formation of the base, whether oleaginous or absorbent, the active ingredient is added to an amount affording the desired concentration.

Creams are oil/water emulsions. They consist of an oil phase (internal phase), comprising typically fixed oils, hydrocarbons and the like, waxes, petroleum, mineral oil and the like and an aqueous phase (continuous phase), comprising water and any water-soluble substances, such as added salts. The two phases are stabilised by use of an emulsifying agent, for example, a surface active agent, such as sodium lauryl sulfite; hydrophilic colloids, such as acacia colloidal clays, veegum and the like. Upon formation of the emulsion, the compound can be added in an amount to achieve the desired concentration.

Gels comprise a base selected from an oleaginous base, water, or an emulsion-suspension base. To the base is added a gelling agent that forms a matrix in the base, increasing its viscosity. Examples of gelling agents are hydroxypropyl cellulose, acrylic acid polymers and the like. Customarily, the compound is added to the formulation at the desired concentration at a point preceding addition of the gelling agent.

The amount of compound incorporated into a topical formulation is not critical; the concentration should be within a range sufficient to permit ready application of the formulation such that an effective amount of the compound is delivered.

### (b) Oral Formulations

The pharmaceutical composition may be adapted for oral delivery. In this regard, the compound can be administered as an oral preparation adapted in such a manner that facilitates delivery of a therapeutically effective concentration of the compound.

The effective dosages of the compound, when administered orally, must take into consideration the diluent, preferably water. The composition preferably contains 0.05% to about 100% by weight active ingredient and more preferably about 10% to about 80% by weight. When the compositions are ingested, desirably they are taken on an empty stomach.

Contemplated for use herein are oral solid dosage forms including tablets, capsules, pills, troches or lozenges, cachets or pellets. Also, liposomal or proteinoid encapsulation may be used to formulate the present compositions. Liposomal encapsulation may be used and the liposomes may be derivatised with various polymers. In general, the formulation will include the compound and inert ingredients that allow for protection against the stomach environment and release of the biologically active material in the intestine.

The location of release may be the stomach, the small intestine (the duodenum, the jejunem, or the ileum), or the large intestine. One skilled in the art has available formulations that will not dissolve in the stomach, yet will release the material in the duodenum or elsewhere in the intestine. Preferably, the release will avoid the deleterious effects of the stomach environment, either by protection of the composition or by release of the compound beyond the stomach environment, such as in the intestine.

To ensure full gastric resistance, a coating impermeable to at least pH 5.0 may be used. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S and Shellac. These coatings may be used as mixed films.

A coating or mixture of coatings that are not intended for protection against the stomach can also be used on tablets. This can include sugar coatings, or coatings that make the tablet easier to swallow. Capsules may consist of a hard shell (such as gelatine) for delivery of dry therapeutic i.e. powder; for liquid forms, a soft gelatine shell may be used. The shell material of cachets could be thick starch or other edible paper. For pills, lozenges, moulded tablets or tablet triturates, moist massing techniques can be used.

One may dilute or increase the volume of the composition with an inert material. These diluents could include carbohydrates, especially mannitol, alpha-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the compound into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatine, orange peel, acid carboxymethyl cellulose, natural sponge and bentonite may all be used. Another form of the disintegrants is insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the composition together to form a hard tablet and include materials from natural products such as acacia, tragacanth, starch and gelatine. Others include methylcellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the compound.

An antifrictional agent may be included in the formulation to prevent sticking during the formulation process. Lubricants may be used as a layer between the compound and the die wall and these can include but are not limited to: stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights and Carbowax 4000 and 6000.

Glidants that might improve the flow properties of the composition during formulation and to aid rearrangement during compression might be added. The glidants may include starch, talc, pyrogenic silica and hydrated silicoaluminate.

To aid dissolution of the compound, a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation either alone or as a mixture in different ratios.

Controlled release formulations may be desirable. The compounds can be incorporated into an inert matrix that permits release by either diffusion or leaching mechanisms i.e., gums. Slowly degenerating matrices may also be incorporated into the formulation. Another form of a controlled release formulation is by a method based on the Oros therapeutic system (Alza Corp.), i.e. the composition is enclosed in a semipermeable membrane which allows water to enter and push the composition out through a single small opening due to osmotic effects. Some enteric coatings also have a delayed release effect.

A mix of materials might be used to provide the optimum film coating. Film coating may be carried out in a pan coater or in a fluidised bed or by compression coating.

The compound can be included in the formulation as fine multiparticulates in the form of granules or pellets of particle size about 1 mm. The formulation of the material for capsule administration could also be as a powder, lightly compressed plugs or even as tablets. The compound could be prepared by compression.

### (c) Injectable Formulations

The compound can also be formulated for parenteral delivery. Pharmaceutical forms suitable for injectable use include sterile aqueous solutions (where water-soluble) or dispersions and sterile powders for the extemporaneous preparation of sterile injectable solutions or dispersion. Alternatively, the compounds of the invention may be encapsulated in liposomes and delivered in injectable solutions to assist their transport across cell membrane. The solution may be a solvent or dispersion medium containing, for example, water, ethanol, polyol (for example, glycerol, propylene glycol and liquid polyethylene glycol and the like), suitable mixtures thereof and vegetable oils. Proper, fluidity may be maintained, for example, by the use of a coating such as lecithin, by the maintenance of the required particle size in the case of dispersion and by the use of superfactants. Prolonged absorption of the injectable compositions can be brought about by the use in the compositions of agents delaying absorption, for example, aluminium monostearate and gelatine.

Sterile injectable solutions may be prepared by incorporating the active compounds in the required amount in an appropriate solvent with various of the other ingredients enumerated above, as required, followed by filtered sterilisation. Generally, dispersions are prepared by incorporating the compound into a sterile vehicle that contains the basic dispersion medium and the other ingredients. In the case of sterile powders for the preparation of sterile injectable solutions, the preferred methods of preparation are vacuum drying and freeze-drying techniques that yield a powder of the compound plus any additional desired ingredient from previously sterile-filtered solution thereof.

Thus, the present invention also provides an injectable, stable, sterile composition comprising a compound of Formula A, or a salt thereof, in a unit dosage form in a sealed container. The compound or salt may be provided in lyophilised form capable of being reconstituted with a suitable pharmaceutically acceptable carrier to form a liquid composition suitable for injection thereof into a subject. The unit dosage form typically comprises from about 10 mg to about 10 grams of the compound or salt thereof. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

### (d) Aerosols

Pharmaceutical compositions are also provided which are suitable for administration as an aerosol, by inhalation. These compositions comprise a solution or suspension of the desired compound or a salt thereof or a plurality of solid particles of the compound or salt. The desired composition may be placed in a small chamber and nebulized. Nebulization may be accomplished by compressed air or by ultrasonic energy to form a plurality of liquid droplets or solid particles comprising the compounds or salts.

The solid particles can be obtained by processing solid compound or a salt thereof, in any appropriate manner known in the art, such as by micronization. Commercial nebulizers are also available to provide liquid droplets of any desired size.

The liquid droplets or solid particles should have a particle size in the range of about 0.5 to about 5 microns, preferably from about 1 to about 2 microns. Most preferably, the size of the solid particles or droplets will be from about 1 to about 2 microns. Such particles or droplets may be dispensed by commercially available nebulisers or by other means known to the skilled person.

When the pharmaceutical composition suitable for administration as an aerosol is in the form of a liquid, the composition will comprise a water-soluble form of the compound or a salt thereof, in a carrier that comprises water. A surfactant may be present which lowers the surface tension of the composition sufficiently to result in the formation of droplets within the desired size range when subjected to nebulization.

In addition, the pharmaceutical composition may also include other agents. For example, preservatives, co-solvents, surfactants, oils, humectants, emollients, chelating agents, dyestuffs, stabilizers or antioxidants may be employed. Water soluble preservatives that may be employed include, but are not limited to, benzalkonium chloride, chlorobutanol, thimerosal, sodium bisulfate, phenylmercuric acetate, phenylmercuric nitrate, ethyl alcohol, methylparaben, polyvinyl alcohol, benzyl alcohol and phenylethyl alcohol. A surfactant may be Tween 80. Other suitable additives include lubricants and slip agents, such as, for example, magnesium stearate, stearic acid, talc and bentonites, substances which promote disintegration, such as starch or crosslinked polyvinylpyrrolidone, binders, such as, for example, starch, gelatin or linear polyvinylpyrrolidone, and dry binders, such as microcrystalline cellulose.

Other vehicles that may be used include, but are not limited to, polyvinyl alcohol, povidone, hydroxypropyl methyl cellulose, poloxamers, carboxymethyl cellulose, hydroxyethyl cellulose, purified water, etc. Tonicity adjustors may be included, for example, sodium chloride, potassium chloride, mannitol, glycerin, etc. Antioxidants include, but are not limited to, sodium metabisulfite, sodium thiosulfate, acetylcysteine, butylated hydroxyanisole, butylated hydroxytoluene, etc. The indications, effective doses, compositions, contraindications, vendors etc, of the compounds in the compositions are available or are known to one skilled in the art. These agents may be present in individual amounts of from about 0.001% to about 5% by weight and preferably about 0.01% to about 2%.

Electrolytes such as, but not limited to, sodium chloride and potassium chloride may also be included in the composition.

Further, the compositions may contain microbial preservatives. Useful microbial preservatives include methylparaben, propylparaben, and benzyl alcohol. The microbial preservative is typically employed when the composition is placed in a vial designed for multidose use.

Excipients which may be used are all the physiologically acceptable solid inert substances, either inorganic or organic in nature. Inorganic substances are, for example, sodium chloride, carbonates, such as calcium carbonate, bicarbonates, aluminium oxides, silicic acids, aluminas, precipitated or colloidal silicon dioxide and phosphates. Organic substances are, for example, sugars, cellulose, foodstuffs and feedstuffs, such as milk powder, animal flours, cereal flours and shredded cereals and starches.

In general, the therapeutically-effective amount of compounds of this invention range from about 5 to 70%, 10-50% or 20-40% by weight. Finally, it will be appreciated that the compositions of the present invention may comprise a plurality of compounds as described herein.

### Method of Treatment

The compounds of the present invention have broad antiparasitic activity, and thus are useful for treating or preventing parasitic infections. Thus, the present invention also provides a method of treating a parasitic infection in a subject comprising the step of administering to the subject an effective amount of a compound of Formula A:

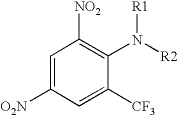

wherein R₁ is C₁-C₅ alkyl, C₃-C₆ branched alkyl, C₄-C₇ cycloalkyl, C₈-C₁₂ fused or bridged polycycloalkyl, or heterocyclic ring, where any of the preceding alkyl, cycloalkyl or heterocyclic ring groups may be singly or multiply substituted with X;

R₂ is H or R₁; and

X is halo, carbonyl, carboxylic acid, carboxylic ester, carboxamide, substituted carboxamide, hydroxy, alkoxy, thioalkyl, sulphoxide, sulphone, sulphonamide, substituted sulphonamide, phenoxy, substituted phenoxy, phenyl, substituted phenyl, amino, substituted amino (including quaternary ammonium salts), N-oxide, imino, 5-7 membered heterocycle, or substituted heterocycle; or a pharmaceutically acceptable salt thereof.

For the purposes of the present invention the term “treating” means ameliorating the deleterious effects of a parasitic infection, inhibiting the onset, growth, or spread of the infection, causing regression of the infection, curing the infection, or otherwise improving the general well-being of an infected subject. This may be achieved by killing or inactivating the parasites or by inhibiting or preventing their reproduction. Preferably, treatment according to the present invention involves the eradication of the parasite and thus cures the infection. For the purposes of the present invention “treating” also encompasses preventing infection or reinfection with a parasite and thus also covers the prophylactic use of the compound.

The parasitic infection may be an infection caused by an endoparasite or an ectoparasite.

Preferably, the parasitic infection is caused by a parasite selected from the group consisting of: trypanosomes; haemoprotozoa and parasites capable of causing malaria; enteric and systemic cestodes including taeniid cestodes; enteric coccidians; enteric flagellate protozoa; filarial nematodes; gastrointestinal and systemic nematodes and hookworms.

More preferably, the trypanosomes are selected from the genera Trypanosoma and Leishmania; the haemoprotazoa are selected from the genera Plasmodium; the taeniid cestodes are selected from the genera Echinococcus; the enteric coccidians are selected from the genera Eimeria and Cryptosporidium; the enteric flagellate protozoa are selected from the genera Giardia; the gastrointestinal and systemic nematodes and hookworms are selected from the genera Amidostomum, Trichostrongylus, Tenorastrongylus, Nippostrongylus, Heligmonina, Boreostrongylus, Ancylostoma; and the filarial nematodes are selected from the genera Wucherieria, Onchocera and Dirofilaria.

Even more preferably, parasitic infection is caused by a parasite selected from the group consisting of: Cryptosporidium andersoni, Cryptosporidium parvum, Cryptosporidium muris, Cryptosporidium hominis, Cryptosporidium wrairi, Cryptosporidium felis, Cryptosporidium canis, Cryptosporidium baileyi, Cryptosporidium meleagridis, Cryptosporidium galli, Cryptosporidium serpentis, Cryptosporidium saurophilum and Cryptosporidium molnari; Echinococcus granulosus, E. multilocularis, E. vogeli, E. oligarthrus; Trypanosome rhodesiense, T. brucei, T. cruzi; G. intestinalis; L. brasiliensis, L. donavani, L. ethiopica L. mexicana L. peruviana, L. tropica, L. major, L. infantum, Plasmodium falciparum, P. humain et simian; Caenorhabditis elegans, Caenorhabditis briggsae, Caenorhabditis drosophilae, Caenorhabditis japonica, Caenorhabditis maupasi, Caenorhabditis plicata, Caenorhabditis remanei, Caenorhabditis sonorae, Caenorhabditis sp. CB5161, Caenorhabditis sp. DF5070, Caenorhabditis sp. PS1010, Caenorhabditis sp. SB341 Caenorhabditis vulgaris, Amidostomum fulicae, A. acutum, Trichostrongylus colubriformis Tenorastrongylus josephi, Nippostrongylus brasiliensis, Nippostrongylus witenbergi, Heligmonina nevoi; Boreostrongylus seurati, Boreostrongylus minutes, Heligmosomoides polygyrus, Wuchereria bancrofti, Onchocerca volvulus, Dirofilaria immitis, Schistosoma mansoni, S. haematobium, S. japonicum, Blastocytis hominis, Pediculus humanis capitis, Onchocera volvulus, Sarcoptes scabei, Trichomonas vaginalis, Toxocaria canis, T. cati and Toxoplasma gondii.

For example, the infection may be caused by a parasite selected from the group consisting of: Caenorhabditis elegans, Trypanosoma rhodesiense, T. brucei, T. cruzi, Leishmania donovani, Plasmodium falciparum, Cryptosporidium, Giardia, or Echinococcus multilocularis.

The present invention also provides a method of treating a parasitic disease in a subject comprising the step of administering to the subject an effective amount of a compound as herein described, wherein said disease is selected from the group comprising: trypanosomiasis, malaria, coccidiosis, leishmaniasis, giardiasis, hookworm infection, Chagas disease, Schistosomiasis (bilharzia), Blastocystosis, cryptosporidiosis, filariasis, head, pubic and body lice infection, ascariasis, onchocerciasis (River blindness), scabies, toxocariasis and toxoplasmosis.

The subjects treated by the method of the present invention may be human but are typically non-human subjects such as animals and in particular livestock and other economically important animals that are farmed or otherwise managed by humans for commercial gain. Thus, subjects encompass any animal, preferably a vertebrate and more preferably a mammal or a bird. Non-mammalian vertebrates include reptiles and freshwater and salt water fish, such as, for example, trout, carp and eels. Also included are insects, such as, for example, honey bees and silk worms. Mammals include, but are not limited to, humans, sport animals, livestock such as cows, horses, sheep, pigs, goats, camels, water buffalo, donkeys, rabbits, fallow deer and reindeer, fur-bearing animals, such as, for example, mink, chinchillas and raccoons. Also included are pets such as dogs, cats and horses and laboratory and experimental animals such as mice, rats, guinea pigs and hamsters. Birds include, but are not limited to, avian livestock such as chickens, geese, turkeys and ducks and pet birds such as pigeons and song birds.

The compound may be administered via any route as deemed appropriate by a suitably qualified practitioner including orally, by inhalation, topically, intramuscularly or intravenously.

As indicated above the compounds of the present invention may also be used to protect a subject against parasite infection, the method comprising the step of administering a prophylactically effective amount of the compound. Such administration may be desired, for example, if the subject is in contact with other subjects which have the infection, or is going to enter an area where the parasite is known to be present.

The effective amounts and dosage requirements (i.e., the amount of each dose, the concentration of the compound used and the frequency of administration) of a subject undergoing treatment of the invention may vary depending on the nature of the compound, the clinical condition of the subject, the diluent, severity and nature of the infection, the response of the subject, the route of delivery or delivery device selected, the side effects and the stability of the compound in the composition. Thus, the skilled person administering the composition comprising a compound of the invention will employ the appropriate preparation containing the appropriate concentration of the compound and select the amount of composition administered, depending upon clinical experience with the subject in question or with similar subjects.

Administration of the compounds of the invention can be carried out in single or multiple doses. As a general proposition, a dosage from about 0.1 to 50, 0.5 to 40 or 1 to 30 mg/kg will have therapeutic efficacy, with still higher dosages potentially being employed for oral and/or aerosol administration. Toxicity concerns at the higher level may restrict intravenous dosages to a lower level, all weights being calculated based upon the weight of the active compound, including the cases where a salt is employed. Typically a dosage from about 0.5 mg/kg to about 5 mg/kg will be employed for intravenous or intramuscular administration. A dosage from about 10 mg/kg to about 50 mg/kg may be employed for oral administration.

The duration of the treatment may be once daily for a period of two to three weeks or until the infection is essentially controlled. Lower doses given less frequently can be used to prevent or reduce the incidence or recurrence of the infection.

The pharmaceutically effective compositions of the present invention may be administered to a subject by any method that leads to delivery of the compound to the site of the infection. The invention is therefore not limited to any one form of delivery in that it includes topical (e.g. application to the skin), systemic (e.g. orally), parenteral (e.g. by intramuscular, intravenous, intraocular or intranasal injection), by inhalation (e.g. using aerosols) or by other means known to the skilled person provided that a sufficient amount of the active compound achieves contact with the site of the endoparasitic infection.

Where the composition contains two or more active agents (e.g. two or more compounds as described herein, or a compound as described herein and another agent), the active agents may be administered as a mixture, as an admixture, in the same composition, in separate compositions, in extended release compositions, liposomes, microcapsules, or any of the previously described embodiments. For example, the composition may be administered topically, or may be injected, or one active agent may be administered topically and the other agent(s) may be injected.

Administration of the composition may be parenteral means (by chemical delivery system or invasive device) to a subject, although other modes of administration may be effective. Parenteral administration is used in appropriate circumstances apparent to the practitioner. Preferably, the compositions are administered in unit dosage forms suitable for single administration of precise dosage amounts.

Parenteral delivery encompasses injection through a variety of routes, such as subcutaneous, intramuscular, intraorbital, intracapsular, intraspinal, intrasternal, intravenous, intranasal etc. The compound to be delivered may be in a depot form. Other parenteral routes of administration and injection sites and forms are also contemplated and are within the scope of the invention.

Additionally, it is also possible to administer the compounds of the present invention topically to an affected part such as the skin. Topical application of compositions of the invention for the treatment or prevention of endoparasitic infections may be as an ointment, gel, eye drops, creams, lotions etc. Preferably a penetrating composition comprising the active compound is used.

Such solutions for use on the skin may be dripped on, brushed on, massaged in, sprinkled on or sprayed on. Pour-on formulations are poured or sprinkled onto limited areas of the skin, the active compound penetrating through the skin and having a systemic action.

The topical composition may further be an in situ gellable aqueous composition. Such a composition comprises a gelling agent in a concentration effective to promote gelling upon contact with the skin, mucous membranes etc. Suitable gelling agents include, but are not limited to, thermosetting polymers such as tetra-substituted ethylene diamine block copolymers of ethylene oxide and propylene oxide (e.g., poloxamine); polycarbophil; and polysaccharides such as gellan, carrageenan (e.g., kappa-carrageenan and iota-carrageenan), chitosan and alginate gums.

The phrase “in situ gellable” as used herein embraces not only liquids of low viscosity that form gels upon contact with the skin, mucous membranes etc, but also more viscous liquids such as semi-fluid and thixotropic gels that exhibit substantially increased viscosity or gel stiffness upon administration to the skin, mucous membranes etc. Indeed, it can be advantageous to formulate a composition of the invention as a gel, to minimize loss of the composition immediately upon administration. Although it is preferred that such a composition exhibit further increase in viscosity or gel stiffness upon administration, this is not absolutely required if the initial gel is sufficiently resistant to dissipation to provide the effective residence time specified herein.

The composition may be also administered as a slow release composition, with a carrier composition such as microspheres, microcapsules, liposomes, etc., as a topical ointment or solution, an intravenous solution or suspension, or in an intraocular injection, as known to one skilled in the art to treat or prevent an endoparasitic infection.

A time-release drug delivery system may be administered by a method such as parenteral administration, topical delivery, oral delivery etc to result in sustained release of the compound over a period of time. The composition may be in the form of a vehicle, such as a micro- or macro-capsule or matrix of biocompatible polymers such as polycaprolactone, polyglycolic acid, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAIB), and other polymers or lipids that may be formulated as microspheres or liposomes.

Delayed or extended release properties may be provided through various compositions of the vehicle (coated or uncoated microsphere, coated or uncoated capsule, lipid or polymer components, unilamellar or multilamellar structure, and combinations of the above, etc.). The composition and loading of microspheres, microcapsules, liposomes, etc. and their delivery are standard techniques known by one skilled in the art.

The invention also provides a pharmaceutical composition comprising: a compound of the present invention or a pharmaceutically acceptable salt thereof, in a biocompatible, biodegradable matrix, for delivery as an implant. When the compound is delivered as an implant, it may be incorporated in any known biocompatible biodegradable matrix as a liquid, or in the form, for example, of a micelle using known chemistry or as microparticles.

Slow or extended-release delivery systems include any of a number of biopolymers (biological-based systems), systems employing liposomes, colloids, resins, and other polymeric delivery systems or compartmentalized reservoirs, can be utilized with the compositions described herein to provide a continuous or long term source of therapeutic compound.

In any slow release device prepared, the active compound is preferably present in an amount of about 10% to 90% by weight of the implant. More preferably, the compound is from about 50% to about 80% by weight of the implant. In a preferred embodiment, the active compound for treatment or prevention of an endoparasitic infection comprises about 50% by weight of the implant. In a particularly preferred embodiment, the active compound comprises about 70% by weight of the implant.

In one form, implants used in the method of the present invention are formulated with particles of the compound entrapped within the bio-erodible polymer matrix.

Release of the agent is achieved by erosion of the polymer followed by exposure of previously entrapped agent particles to the vitreous, and subsequent dissolution and release of agent. The release kinetics achieved by this form of drug release are different than that achieved through compositions which release drug through polymer swelling, such as with hydrogels such as methylcellulose. In that case, the drug is not released through polymer erosion, but through polymer swelling, which releases drug as liquid diffuses through the pathways exposed. The parameters which determine the release kinetics include the size of the drug particles, the water solubility of the drug, the ratio of drug to polymer, the method of manufacture, the surface area exposed, and the erosion rate of the polymer.

Exemplary biocompatible, non-biodegradable polymers of particular interest include polycarbamates or polyureas, particularly polyurethanes, polymers which may be cross-linked to produce non-biodegradable polymers such as cross-linked poly(vinyl acetate) and the like. Also of particular interest are ethylene-vinyl ester copolymers having an ester content of 4 to 80% such as ethylene-vinyl acetate (EVA) copolymer, ethylene-vinyl hexanoate copolymer, ethylene-vinyl propionate copolymer, ethylene-vinyl butyrate copolymer, ethylene-vinyl pentantoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer.

Additional exemplary naturally occurring or synthetic non-biodegradable polymeric materials include poly(methylmethacrylate), poly(butylmethacrylate), plasticized poly(vinylchloride), plasticized poly(amides), plasticized nylon, plasticized soft nylon, plasticized poly(ethylene terephthalate), natural rubber, silicone, poly(isoprene), poly(isobutylene), poly(butadiene), poly(ethylene), poly(tetrafluoroethylene), poly(vinylidene chloride), poly(acrylonitrile, cross-linked poly(vinylpyrrolidone), poly(trifluorochloroethylene), chlorinated poly(ethylene), poly(4,4′-isopropylidene diphenylene carbonate), vinylidene chloride-acrylonitrile copolymer, vinyl chloridediethyl fumarate copolymer, silicone, silicone rubbers (especially the medical grade), poly(dimethylsiloxanes), ethylene-propylene rubber, silicone-carbonate copolymers, vinylidene chloride-vinyl chloride copolymer, vinyl chloride-acrylonitrile copolymer, vinylidene chloride-acrylonitrile copolymer, poly(olefins), poly(vinyl-olefins), poly(styrene), poly(halo-olefins), poly(vinyls), poly(acrylate), poly(methacrylate), poly(oxides), poly(esters), poly(amides), and poly(carbonates)

Diffusion of the compound from the implant may also be controlled by the structure of the implant. For example, diffusion from the implant may be controlled by means of a membrane affixed to the polymer layer comprising the compound. The membrane layer will be positioned intermediate to the polymer layer comprising the compound and the desired site of therapy. The membrane may be composed of any of the biocompatible materials indicated above and may vary with the compound employed, the presence of agents in addition to the compound present in the polymer, the composition of the polymer comprising the compound, the desired rate of diffusion and the like. For example, the polymer layer will usually comprise a very large amount of compound and will typically be saturated. Such saturated polymers may generally release the compound at a very high rate. In this situation, the release of the compound may be slowed by selecting a membrane which is of a lower compound permeability than the polymer. Due to the lower permeability of the membrane, the compound will remain concentrated in the polymer and the overall rate of diffusion will be determined by the permeability of the membrane. Therefore, the rate of release of the compound from the implant is reduced, providing for a more controlled and extended delivery to the site of therapy.

The compositions of the present invention may alternatively be delivered with the aid of shaped articles containing the active compound, such as, for example, strips, plates, tapes, collars, ear tags, limb bands or marking devices.

The skilled reader will appreciate that the duration over which any of the compositions used in the method of the invention will dwell in the tissues of the subject will depend, inter alia, on such factors as the pharmacological properties of the compounds employed in the composition, the concentration of the compound employed, the bioavailability of the compound, the infection to be treated, the mode of administration and the preferred longevity of the treatment.

Where that balance is struck will often depend on the longevity of the effect required and the particular parasite infection being treated.

The frequency of treatment according to the method of the invention is determined according to the infection being treated, the deliverable concentration of the active compound and the method of delivery. Once a therapeutic result is achieved, the compound can be tapered or discontinued. Occasionally, side effects warrant discontinuation of therapy. In general, an effective amount of the compound is that which provides either subjective relief of symptoms or an objectively identifiable improvement as noted by a suitably qualified practitioner.

### Use of Compounds

The present invention further provides for the use of a compound of Formula A:

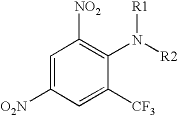

wherein R₁ is C₁-C₅ alkyl, C₃-C₆ branched alkyl, C₄-C₇ cycloalkyl, C₈-C₁₂ fused or bridged polycycloalkyl, or heterocyclic ring, where any of the preceding alkyl, cycloalkyl or heterocyclic ring groups may be singly or multiply substituted with X;

R₂ is H or R₁; and

X is halo, carbonyl, carboxylic acid, carboxylic ester, carboxamide, substituted carboxamide, hydroxy, alkoxy, thioalkyl, sulphoxide, sulphone, sulphonamide, substituted sulphonamide, phenoxy, substituted phenoxy, phenyl, substituted phenyl, amino, substituted amino (including quaternary ammonium salts), N-oxide, imino, 5-7 membered heterocycle, or substituted heterocycle; or a pharmaceutically acceptable salt thereof to prepare a medicament to treat a parasitic infection in a subject.

The compound being used to prepare the medicament is preferably able to treat a parasitic infection caused by an endoparasite or an ectoparasite.

Preferably, the compound being used to prepare the medicament is able to treat a parasitic infection caused by a parasite selected from the group consisting of: trypanosomes; haemoprotozoa and parasites capable of causing malaria; enteric and systemic cestodes including taeniid cestodes; enteric coccidians; enteric flagellate protozoa; filarial nematodes; gastrointestinal and systemic nematodes and hookworms.

More preferably, the compound being used to prepare the medicament is able to treat a parasitic infection caused by trypanosomes selected from the genera Trypanosoma and Leishmania; haemoprotazoa selected from the genera Plasmodium; taeniid cestodes selected from the genera Echinococcus; enteric coccidians selected from the genera Eimeria and Cryptosporidium; enteric flagellate protozoa selected from the genera Giardia; gastrointestinal and systemic nematodes and hookworms selected from the genera Amidostomum, Trichostrongylus, Tenorastrongylus, Nippostrongylus, Heligmonina, Boreostrongylus, Ancylostoma; and filarial nematodes selected from the genera Wucherieria, Onchocera and Dirofilaria.

Even more preferably, the compound being used to prepare the medicament is able to treat a parasitic infection caused by a parasite selected from the group consisting of: Cryptosporidium andersoni, Cryptosporidium parvum, Cryptosporidium muris, Cryptosporidium hominis, Cryptosporidium wrairi, Cryptosporidium felis, Cryptosporidium canis, Cryptosporidium baileyi, Cryptosporidium meleagridis, Cryptosporidium galli, Cryptosporidium serpentis, Cryptosporidium saurophilum and Cryptosporidium molnari; Echinococcus granulosus, E. multilocularis, E. vogeli, E. oligarthrus; Trypanosome rhodesiense, T brucei, T. cruzi; G. intestinalis; L. brasiliensis, L. donavani, L. ethiopica L. mexicana L. peruviana, L. tropica, L. major; L. infantum, Plasmodium falciparum, P. humain et simian; Caenorhabditis elegans, Caenorhabditis briggsae, Caenorhabditis drosophilae, Caenorhabditis japonica, Caenorhabditis maupasi, Caenorhabditis plicata, Caenorhabditis remanei, Caenorhabditis sonorae, Caenorhabditis sp. CB5161, Caenorhabditis sp. DF5070, Caenorhabditis sp. PS1010, Caenorhabditis sp. SB341 Caenorhabditis vulgaris, Amidostomum fulicae, A. acutum, Trichostrongylus colubriformis Tenorastrongylus josephi, Nippostrongylus brasiliensis, Nippostrongylus witenbergi, Heligmonina nevoi; Boreostrongylus seurati, Boreostrongylus minutes, Heligmosomoides polygyrus, Wuchereria bancrofti, Onchocerca volvulus, Dirofilaria immitis, Schistosoma mansoni, S. haematobium, S. japonicum, Blastocytis hominis, Pediculus humanis capitis, Onchocera volvulus, Sarcoptes scabei, Trichomonas vaginalis, Toxocaria canis, T. cati and Toxoplasma gondii.

For example, the parasitic infection may be caused by a parasite selected from the group consisting of: Caenorhabditis elegans, Trypanosoma rhodesiense, T. brucei, T. cruzi, Leishmania donovani, Plasmodium falciparum, Cryptosporidium, Giardia, or Echinococcus multilocularis.

The invention also provides for the use of a compound as herein described for the preparation of a medicament to treat a parasitic disease in a subject, wherein the disease is selected from the group comprising: trypanosomiasis, malaria, coccidiosis, leishmaniasis, giardiasis, hookworm infection, Chagas disease, Schistosomiasis (bilharzia), Blastocystosis, cryptosporidiosis, filariasis, head, pubic and body lice infection, ascariasis, onchocerciasis (River blindness), scabies, toxocariasis and toxoplasmosis.

The present invention also provides for the use of a compound of Formula A:

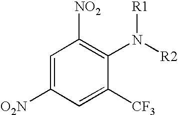

wherein R₁ is C₁-C₅ alkyl, C₃-C₆ branched alkyl, C₄-C₇ cycloalkyl, C₈-C₁₂ fused or bridged polycycloalkyl, or heterocyclic ring, where any of the preceding alkyl, cycloalkyl or heterocyclic ring groups may be singly or multiply substituted with X;

R₂ is H or R₁; and

X is halo, carbonyl, carboxylic acid, carboxylic ester, carboxamide, substituted carboxamide, hydroxy, alkoxy, thioalkyl, sulphoxide, sulphone, sulphonamide, substituted sulphonamide, phenoxy, substituted phenoxy, phenyl, substituted phenyl, amino, substituted amino (including quaternary ammonium salts), N-oxide, imino, 5-7 membered heterocycle, or substituted heterocycle; or a pharmaceutically acceptable salt thereof to prepare a medicament to protect a subject against a parasite infection.

The invention also provides for the use of a compound as herein described for the preparation of a medicament to protect against a parasitic disease in a subject, wherein the disease is selected from the group comprising: trypanosomiasis, malaria, coccidiosis, leishmaniasis, giardiasis and hookworm infection.

The present invention further provides for the use of a compound as described herein or a pharmaceutically acceptable salt thereof in the manufacture of a medicament for the treatment or prophylaxis of a parasitic infection in a subject in need thereof.

The present invention further provides for the use of a compound as described herein or a pharmaceutically acceptable salt thereof in the manufacture of a medicament for the treatment or prophylaxis of a disease selected from the group comprising: trypanosomiasis, malaria, coccidiosis, leishmaniasis, gairdiasis and hookworm infection.

### General

To the extent that any compounds disclosed in Japanese patent application 76-112673 are included in the scope of the compound claims herein, they are hereby disclaimed. However, applicant reserves their rights to claim the use of compounds within JP 76-112673 and/or particular formulations of these compounds.

Those skilled in the art will appreciate that the invention described herein is susceptible to variations and modifications other than those specifically described. The invention includes all such variation and modifications. The invention also includes all of the steps, features, compositions and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

The present invention is not to be limited in scope by the specific embodiments described herein, which are intended for the purpose of exemplification only. Functionally equivalent products, compositions and methods are clearly within the scope of the invention as described herein.

The invention described herein may include one or more range of values (eg size, concentration etc). A range of values will be understood to include all values within the range, including the values defining the range, and values adjacent to the range which lead to the same or substantially the same outcome as the values immediately adjacent to that value which defines the boundary to the range.

For the purposes of the present invention an “endoparasite” is a parasite that has at least one lifecycle stage that lives intracellularly and/or within the tissues of its host. Such parasites are dependent on at least one gene or its product from that host to complete their own life-cycle.

A “pharmaceutically acceptable carrier” is a material that is not biologically or otherwise undesirable, i.e., the material can be administered to an individual along with the active agent(s) without causing unacceptable biological effects or interacting in a deleterious manner with any of the other components of the pharmaceutical composition in which it is contained.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the description of the invention and apply throughout. Unless otherwise defined, all other scientific and technical terms used herein have the same meaning as commonly understood to one of ordinary skill in the art to which the invention belongs.

### EXAMPLES

Further features of the present invention are more fully described in the following non-limiting Examples. This detailed description is included solely for the purposes of exemplifying the present invention. It should not be understood in any way as a restriction on the broad description of the invention as set out above.

### Example 1

### 1-Morpholino-2,4-dinitro-6-(trifluoromethyl)benzene

Morpholine (3.26 mL, 37.4 mmol) was added to a solution of 2-chloro-3,5-dinitrobenzotrifluoride (1.74 g, 6.43 mmol) in dichloromethane (25 mL). The mixture was stirred at room temperature for 2 days and then left to stand for two days. The mixture was then washed with 1M hydrochloric acid solution (2×25 mL) followed by saturated sodium bicarbonate solution (2×25 mL) and then water (25 mL). The organic layer was separated, dried over magnesium sulphate and concentrated to give the crude product as orange oil that quickly crystallised (2.00 g). The crude product was purified by medium pressure chromatography (Flashmaster II chromatography system) using a 20 g silica gel column. A mixture of hexanes and ethylacetate were used as eluents. The purest fractions were combined, the solvent was removed in vacuo and the residue was dried under high vacuum for one and a half days to give the desired product as a yellow powder (593 mg, 29%) mp 102-103° C.

### Example 2

### 1-Thiomorpholino-2,4-dinitro-6-(trifluoromethyl)benzene

To a solution of 2-chloro-3,5-dinitrobenzotrifluoride (220 mg, 0.81 mmol) in dichloromethane (15 ml) was added thiomorpholine (0.21 mL, 2.22 mmol). The mixture was stirred at room temperature for approximately 42 hours. The mixture was then washed with water (3×20 mL) and saturated sodium bicarbonate solution (20 mL). The organic layer was separated, dried over magnesium sulphate and concentrated. The crude product was purified by medium pressure chromatography (Flashmaster II chromatography system) over silica gel using gradient elution from 100% hexanes to 100% dichloromethane to afford the product as a yellow powder (201 mg, 74%) mp 120-121° C.

### Example 3

### 1-(4-Acetyl-1-piperazinyl)-2,4-dinitro-6-(trifluoromethyl)benzene

To a solution of 2-chloro-3,5-dinitrobenzotrifluoride (203 mg, 0.75 mmol) in dichloromethane (15 mL) was added 1-acetylpiperazine (302 mg, 2.36 mmol) and the mixture was stirred at room temperature for one week. The mixture was then washed with water (3×20 mL) and saturated sodium bicarbonate solution (20 mL). The organic layer was separated, dried over magnesium sulphate and concentrated. The crude product was purified by medium pressure chromatography (Flashmaster II chromatography system) over silica gel using mixtures of hexanes and ethyl acetate as eluents to give the pure product as a bright yellow powder (166 mg, 61%) mp 126° C.

### Example 4

### 1-(4-Ethyl-1-piperazinyl)-2,4-dinitro-6-(trifluormethyl)benzene

A solution of 2-chloro-3,5-dinitrobenzotrifluoride (200 mg, 0.74 mmol) in dichloromethane (2 mL) was added to 1-ethylpiperazine (0.28 mL, 2.22 mmol) followed by a further 2 mL of dichloromethane. The mixture was stirred at room temperature for one hour. Water (5 mL) was then added and stirring continued for a further 10 minutes. The organic layer was then washed two more times in the same manner (5 mL water, stirring for 10 minutes) and dried over magnesium sulphate. The solvent was removed and the crude product was purified by medium pressure chromatography (Flashmaster II chromatography system) using mixtures of hexanes and ethyl acetate as eluents to afford the desired product as an orange solid (245 mg, 95%) mp 91-92° C.; δ_{H} (300 MHz, CDCl₃)

### Example 5

### 1-(4-(2-Pyrimidyl)-1-piperazinyl)-2,4-dintiro-6-(trifluoromethyl)benzene

A solution of 2-chloro-3,5-dinitrobenzotrifluoride (200 mg, 0.74 mmol) in dichloromethane (2 mL) was added to 1-(2-pyrimidyl)piperazine (0.125 g, 0.76 mmol) and triethylamine (0.2 mL, 1.4 mmol) followed by a further 2 mL of dichloromethane. The mixture was stirred at room temperature for one hour. Water (5 mL) was then added and stirring continued for a further 10 minutes. The organic layer was then washed two more times in the same manner (5 mL water, stirring for 10 minutes) and dried over magnesium sulphate. The solvent was removed and the crude product was purified by medium pressure chromatography (Flashmaster II chromatography system) using mixtures of hexanes and ethyl acetate as eluents affording the unreacted 2-chloro-3,5-dinitrobenzotrifluoride (87 mg) and the desired product (100 mg, 60% based on the amount of starting material consumed) a yellow powder mp 129.5-132° C.

### Example 6

### N-ethylmorpholino-2,4-dinitro-6-(trifluoromethyl)aniline

A solution of 2-chloro-3,5-dinitrobenzotrifluoride (200 mg, 0.74 mmol) in dichloromethane (2 mL) was added to 4-(2-aminoethyl)morpholine (0.096 mL, 0.74 mmol) and triethylamine (0.2 mL, 1.4 mmol) followed by a further 2 mL of dichloromethane. The mixture was stirred at room temperature for one hour. Water (5 mL) was then added and stirring continued for a further 10 minutes. The organic layer was then washed two more times in the same manner (5 mL water, stirring for 10 minutes) and dried over magnesium sulphate. The solvent was removed and the crude product was purified by medium pressure chromatography (Flashmaster II chromatography system) using mixtures of hexanes and ethyl acetate as eluents to yield the desired product as a yellow powder (171 mg, 63%) mp 152.5-154° C.

### Example 7

### 1-(4-(1-pyrrolidinyl)-1-piperadinyl)-2,4-dinitro-6-(trifluoromethyl)benzene

A solution of 2-chloro-3,5-dinitrobenzotrifluoride (200 mg, 0.74 mmol) in dichloromethane (2 mL) was added to 4-(1-pyrrolidinyl)piperidine (0.118 g, 0.76 mmol) and triethylamine (0.2 mL, 1.4 mmol) followed by a further 2 mL of dichloromethane. The mixture was stirred at room temperature for one hour. Water (5 mL) was then added and stirring continued for a further 10 minutes. The organic layer was then washed two more times in the same manner (5 mL water, stirring for 10 minutes) and dried over magnesium sulphate. The solvent was removed and the crude product was purified by medium pressure chromatography (Flashmaster II chromatography system) using mixtures of hexanes and ethyl acetate as eluents to yield the desired product as an orange powder (113 mg, 39%) mp 82-83.5° C.

### Example 8

### Screening Against Giardia

### Materials/Methods

### A. Cell Culture/Initial Screen

Giardia were grown in flat sided tubes (Nunc), with all air excluded, tightly sealed at 37° C. in maintenance media in accordance with known methods.

Drugs were screened against Giardia in 96 well plates at 100 μM concentrations. The final liquid volume per well is 200 μl. IC₅₀ Albendazole controls were included on each plate (400 nM). The Giardia was exposed to the drug for 48 hours in total. The alamarBlue calorimetric metabolic/growth indicator alamarBlue (Australian Laboratory Sciences or Jamar Diagnostics) was added about 5-6 hours before the plate was read for metabolism and colour change at 48 hours.
1. For each screening plate, remove and discard media from one tube of Giardia at monolayer. Replace with new media and place sealed tubes in fridge/freezer until Giardia have detached from tube wall (this can be helped by rolling the tube vigorously between the palms of your hands). This should leave Giardia at between 1×10⁶/ml and 6×10⁵/ml. Once diluted with drug the final concentration of Giardia should be around 3×10⁵/ml.
2. Drugs to be screened are pre-diluted to a concentration of 200 μM. A volume sufficient to allow 100 μl to be dispensed to each screening and drug blank well is prepared (1:50 dilution of 10 mM to give 700 μl final volume i.e. 14 μl drug+686 μl media).
3. Add 100 μl of 200 μM drug to the appropriate screening wells and drug blank wells on the screening plate
4. Dilute 80 μM Albendazole stock 1:100 to give 800 nM (7 μl ABZ+693 μl Media) and dispense 100 μl of this dilution to each of the ABZ control wells.
5. Add 200 μl of warm media to the Media only and Media+alamarBlue wells.
6. Add 100 μl of warm media to the Drug+Media blank, P1c±0 only and ABZ Control wells.
7. Once the Giardia have detached from the tube wall, add 100 μl of Giardia suspension to all wells. The addition of Giardia suspension completes the dilution of the drug screening and control wells to 100 μM and 400 nM respectively.
8. Tape up around lid and incubate plate in candle box in 37° C. room for 48 h.
9. After about 42 hours add 20 μl of alamarBlue (aB) to all of the drug screening wells, the P1c10 only wells, the ABZ control wells and the Media+aB wells. (DO NOT add the aB to the Drug+Media blank wells or the Media only wells.)
10. Re-tape the plates and return immediately to the candle box for a further 6 hours.
11. Once 48 hours has elapsed, the plate is read on a Biorad microplate reader at 570 nm and 630 nm.

The data is used to calculate the % Inhibition for each drug and the ABZ control. Those drugs giving over 50% inhibition go on to have a dose response curve assay.

### B. Dose/Response

As with screening, dose response curves (DRC) against Giardia are performed in 96 well plates with 3 curves per plate. Initial curves start at 100 μM and decrease to 0.1 μM. The curve may be repeated using a tenfold decrease in concentration range (10 μM-0.01 μM) if the IC₅₀ is demonstrated to be close to the lowest concentration. All aspects of plate set up remain the same as with the screening plates i.e. 200 μl final volume, ABZ controls included on each plate, 48 hr exposure, alamarBlue added about 5-6 hours before the plate is read for metabolism and colour change at 48 hours.
1. For each DRC plate to be set up, remove and discard media from one tube of Giardia at monolayer. Replace with new media and place sealed tubes in fridge/freezer until Giardia have detached from tube wall (this can be helped by rolling the tube vigorously between the palms of your hands). This should leave the Giardia at between 1×10⁶/ml and 6×10⁵/ml. Once diluted with drug, the final concentration of Giardia should be around 3×10⁵/ml.
2. Drugs are pre-diluted in a 48 well plate. As with the screening plates, a volume sufficient to allow 100 μl to be dispensed to each test and drug blank well is prepared. Dilute 80 μM Albendazole stock 1:100 to give 800 nM (7 μl ABZ+693 μl Media).
3. Add 200 μl of warm media to the Media only and Media+aB wells.
4. Add 100 μl of warm media to the Drug+Media blank wells, P1c10 only and ABZ Control wells.
5. Add 100 μl of drug dilution to the appropriate test wells and drug blank well on the DRC plate.
6. Once Giardia have detached from the tube wall, add 100 μl of Giardia suspension to all wells excluding Drug+Media Blank wells. The addition of the Giardia suspension completes the dilution of the drug screening and control wells to 100 μM and 400 nM respectively.
7. Tape up around lid and incubate plate in candle box in 37° C. room for 42 hours.
8. After about 42 hours add 25 μl of aB to all of the drug screening wells, the P1c10 only wells, the ABZ control wells and the Media+aB wells. (DO NOT add the aB to the Drug+Media blank wells or the Media only wells.)
9. Re-tape the plates and return immediately to the candle box for a further 6 hours.
10. Once a total of 48 hours has elapsed, the plate is read on a Biorad microplate reader at 570 nm and 630 nm.

### Results

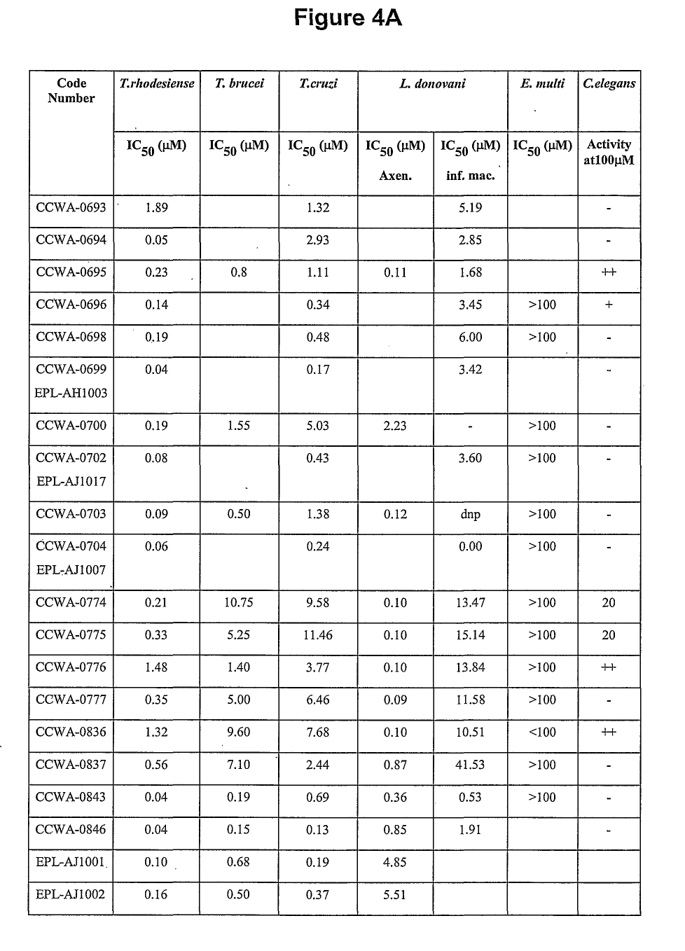

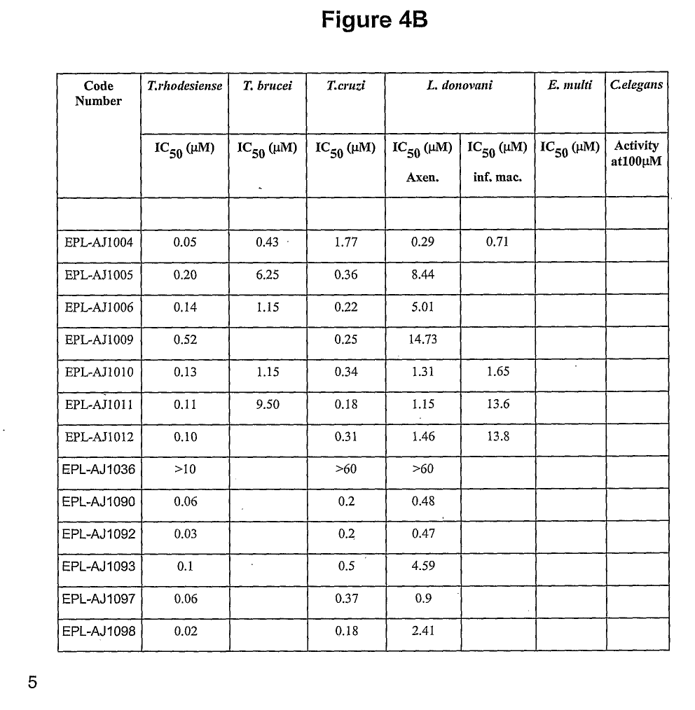

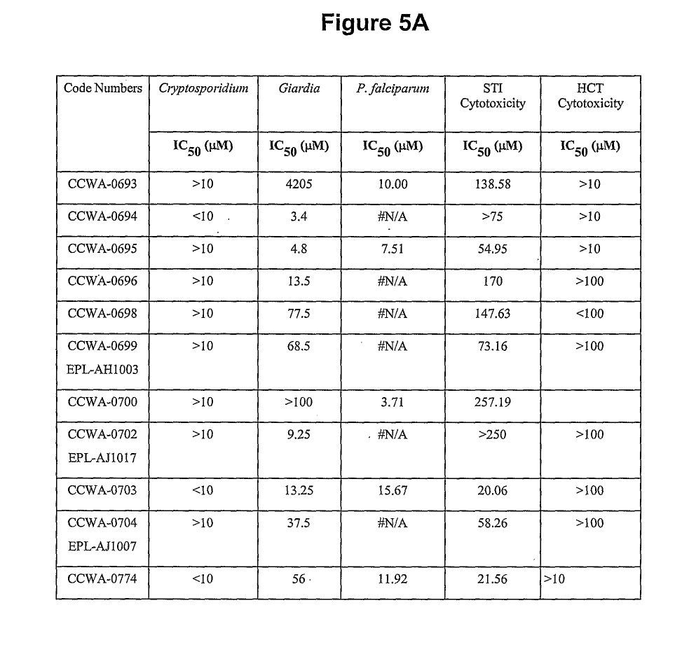

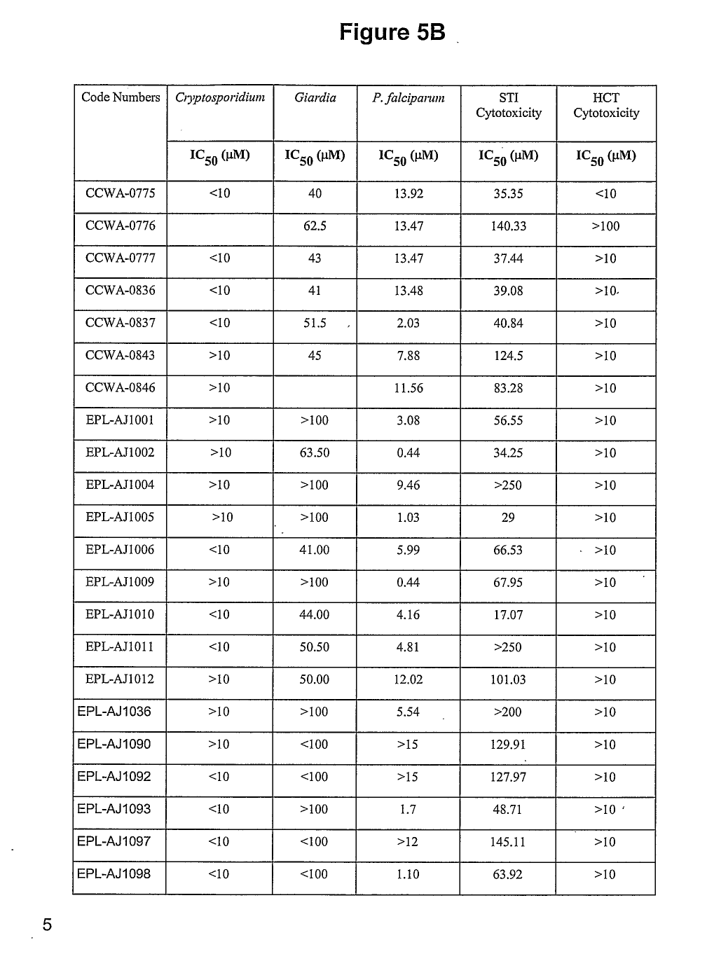

FIGS. 4A and 4B set out the results of the screens against Giardia

### Example 9

### Screening Against Cryptosporidium

### Materials/Methods

### A. Cell Culture/Initial Screen

Cryptosporidium was grown on a HCT-8 cell monolayer in maintenance media, at 37° C. and 5% CO₂ in accordance with known methods [Please provide reference]

Compounds showing no cytotoxic effect on HCT-8 cells at 100 μM are screened against Cryptosporidium infected monolayers of HCT-8 cells at 100 μM. Those showing a cytotoxic effect on HCT-8 cells at 100 μM but not at 10 μM are screened against Cryptosporidium infected monolayers of HCT-8 cells at 10 μM. Compounds are added to infected cell monolayers 12 to 24 hrs post infection.
1. Add 5 μl of DMSO to −ve and +ve controls.
2. Dilute 10 mM stock 1:10 with DMSO (10 μl compound+90 μl DMSO) in 96 well plates.
3. Add 5 μl of 1 mM compound for a final concentration of 10 μM.
4. Incubate plate at 37° C. for 48 hr
5. After incubation examine microscopically and note any signs of monolayer disturbance or cytotoxicity.
6. Remove media, wash monolayers with 500 μl PBS.
7. Check monolayers are still intact, only continue with those wells containing an intact monolayer
8. Add 70 μl of Trypsin and incubate at 37° C. for 10 min.
9. Freeze (−20° C.) plates until ready to complete DNA extraction.

### B. Dose Response

Compounds showing anti-cryptosporidial activity by single concentration screening are further examined to produce a dose response curve and consequently ascertain an IC₅₀. The highest concentration used is dependent on the concentration of the compound when screened (100 μM or 10 μM) and the degree of difference in threshold cycle (C_{T}) between the screened concentration and the +ve control. If the difference between C_{T} is minimal, the screening concentration is included in the dose response curve as the highest concentration. If the difference in C_{T} is high, indicating almost complete inhibition, the highest concentration used for the dose response curve should be a ten-fold reduction of that screened.

HCT-8 monolayers in 48 well culture plates should include 6+ve control wells, these will be used to generate a standard curve. Also included are 2−ve controls.

The compound is tested at 4 concentrations in quadruplet, decreasing serially by a factor of 10. Compounds are added to infected cell monolayers 12 to 24 hrs post infection.
1. Add 5 μl of DMSO to −ve and +ve controls.
2. Add 5 μl of serially diluted compound to appropriate well.
3. Incubate plate at 37° C. in candle box for 48 hr
4. After incubation examine microscopically and note any signs of monolayer disturbance or cytotoxicity.
5. Remove media, wash monolayers with 500 μl PBS.
6. Check monolayers are still intact, only continue with those wells containing an intact monolayer
7. Add 70 μl of Trypsin and incubate at 37° C. for 10 min.
8. Freeze (−20° C.) plates until ready to complete DNA extraction.
   C. DNA Extraction for QPCR from Drug Screening and Dose Response Curve Plates
1. Remove plates from −20° C. and add 100 μl of 1×PCR buffer (For 10× buffer: 500 mM KCL, 100 mM Tris-HCL, pH 8.3).
2. Repeat pipette to mix trypsin, cells and PCR buffer suspension and remove to labelled microfuge tube.
3. Wash well with a further 200 μl of 1×PCR buffer and pool with previous wash in appropriate microfuge tube.
4. Spin at 14000 rpm for 20 min.
5. Remove and discard supernatant (take care to avoid losing pellet).
6. Spin at 14000 rpm for 5 min.
7. Remove and discard supernatant (take care to avoid losing pellet).
8. Resuspend pellet in 30 μl of 1×PCR buffer.
9. Boil for 30 min.
10. Store at −20° C., repeat last spin step upon thawing before use.

### D. Taqman PCR of CCWA Screening and Dose Response Plates

### (i) Screening Plate Analysis

Primers and Probe

| | |
|---|---|
| RH Forward | 5′ AAGAAGGCCGTGTTGGCTTA 3′ |
| RH Reverse | 5′ GGGATTCAGCCCACCAGAAT 3′ |
| Probe | 5′ TTCTGAGCTTTCTTGTGCAGTTTGTGGTACA 3′ |

Product size 85 bp
Primer Concentration 10 pmol per reaction
Probe Concentration 1.25 pmol per reaction

### (ii) PCR Mastermix:

per 25 μl reaction

| | |
|---|---|
| 2.5 μl | 10x Reaction Buffer |
| 4.5 μl | MgCl₂ |
| 1.0 μl | DNTP's |
| 0.1 μl | Tth+ |
| 1.0 μl | RH Forward |
| 1.0 μl | RH Reverse |
| 1.0 μl | Probe |
| 8.9 μl | H₂O (Milli-Q) |
| 5.0 μl | DNA |

(iii) PCR Amplification

Each run takes approximately 92 minutes to complete

| Cycles | Temperature | Time | |
|---|---|---|---|
| 1× | 95° C. | 2 | min |
| 40× | 94° C. | 20 | secs |
| | 60° C. | 60 | secs |

### (iv) PCR Analysis

PCR analysis is carried out using the Rotor-Gene software (Cobeft)

Anti-cryptosporidial activity is assessed by the direct comparison of the Threshold cycle (C_{T}) for each of the drug treated infections against the +ve controls.

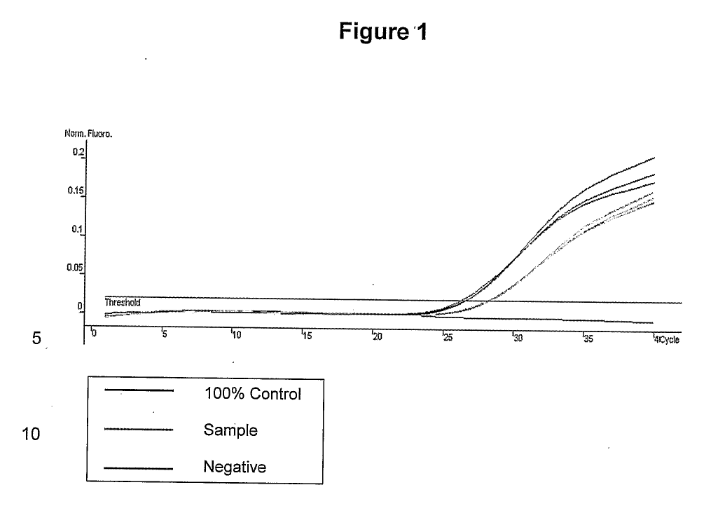

The threshold is set so that the PCR −ve and cell culture −ve controls give a C_{T} of 40 and the +ve controls had reached the exponential phase of amplification (see FIG. 1). The results are updated to set the same threshold for all reactions included in that plate and the threshold is noted.

FIG. 1 shows an example of an amplification curve for a cryptosporidial infection in the presence of a compound. In this plot the threshold has been set at a Δ_{Rn} of 0.02. At this Δ_{Rn} the amplification curves for the negative controls have a C_{T} of 40. (The amplification plots for these negative controls may show a slight upward slope towards the final cycles, this is most likely due to probe degeneration). In this case the compound has clearly had an inhibitory effect on the infection when compared with the +ve control although the inhibition has not been complete.

Those compounds showing a reasonably higher C_{T} (i.e. more that 4 cycles) than the +ve undergo further investigation to determine a dose response curve and consequently an IC₅₀.

### E. Dose Response Curve Plate Analysis

### (i) Standard Curve Preparation for PCR

The 6+ve controls are included where possible on an earlier PCR run to determine the 4 most consistent controls to be used to formulate the standard curve. DNA from the 4 chosen +ve control wells are serially diluted 1:2, giving 100%, 50%, 25%, 12.5% and 6.25% concentrations of DNA.

### (ii) PCR Amplification of Dose Response Plates

PCR plates were set up in the following order for ease in later analysis;

### 4×100% Std; 4×50% Std; 4×25% Std; 4×12.5% Std; 4×6.25% Std

2×−ve; 1×PCR+ve control, 1×PCR −ve control
4×Drug1at10 μM; 4×Drug1at0.1 μM; 4×Drug1at0.1 μM; 4×Drug1at0.01 μM
4×Drug2at10 μM; 4×Drug2at1 μM; 4×Drug2 at0.1 μM; 4×Drug2 at0.01 μM

### 4×Drug Y at 10 μM; 4×Triflu at 0.01 μM

PCR amplification was carried out under the same master mix and cycling conditions as for drug screening.

(iii) Analysis of PCR results for Dose Response Plates

Analysis of PCR results and composition of standard curves is carried out using the Rotor-Gene (Corbett) software.
Standard Curve Composition

Standard curves are formulated using the Rotor Gene Software. Due to the capacity of Cryptosporidium to exhibit occasional, unexplainable but vast variations in culture characteristics it is sometimes necessary to edit the standard curve (although the need for this is less common with the use of 6 pre-checked +ve controls). This is done by excluding the outlying point. The degree of editing should be taken into account when assessing the reliability of the curve. In formulating the standard curve, the threshold is set for all of the reactions at the point that gives the best correlation coefficient.

The Rotor-Gene (Corbett) software program will automatically work out a standard curve based on the information provided. The “Auto-find Threshold” function automatically finds the threshold that provides the best correlation coefficient. It is important to make sure that this threshold still lies in the exponential phase of amplification.

The information from the software can then be exported into a program such as Excel and the average C_{T} can be calculated for each of the drug concentrations and for the standards. Using the averaged C_{T} the percentage inhibition is worked out using the following equation:

conc=10̂(−0.361*CT+12.429)

### Results

The results of the screens against Cryptosporidium are set out in Tables 3A, 3B, 4A and 4B.

### Example 10

### Screening Against Trypanosomes

### Materials/Methods

### A. Cell Culture/Initial Screening

Trypanosome were grown in HMI-9 media with blood stream form-supporting factors (BSF-S factors) and 20% Fresh Horse Serum.

Drugs are screened against Trypanosomes in 96 well plates at 100 μM concentrations. The final liquid volume per well is 100 μl. IC₅₀ Diminazene controls are included on each plate (10 ng/ml). The Trypanosomes are exposed to the drug for 72 hours in total. The alamarBlue is added after 48 hours incubation allowing 24 hours for metabolism and colour change prior to the plate being read at 72 hours.
1. Using a haemocytometer, count a 10 ml culture of T. brucei at optimum growth (i.e. high in numbers without any short and stumpy forms apparent). Calculate the dilution factor needed to achieve a concentration of organisms at about 1×10⁵/ml. The Trypanosomes will be diluted a further 1:2 during plate set up, so that the final concentration of organisms should be about 5×10⁴/ml.
2. Drugs to be screened are pre-diluted in a 24 or 48 well plate to a concentration of 200 μM. A volume sufficient to allow 50 μl to be dispensed to each screening and drug blank well is prepared (1:50 dilution of 10 mM to give 500 μl final volume ie. 10 μl drug+490 μl media).
3. Add 50 μl of 200 μM drug to the appropriate screening wells and drug blank wells on the screening plate.
4. Dilute 1 μg/ml Diminazene stock 1:50 to give 20 nM (8 μl Dimin.+396 μl Media) and dispense 50 μl of this dilution to each of the Dimin. control wells.
5. Add 100 μl of warm media to the Media only and Media+aB wells.
6. Add 50 μl of warm media to the Drug+Media blank, Tryps only and Dimin. Control wells.
7. Dilute the 10 ml culture of Trypanosomes previously counted to achieve 1×10⁵/ml with warm media. Add 50 μl of Tryps suspension to all wells except the media only controls. The addition of Tryps suspension completes the dilution of the drug screening and control wells to 100 μM and 10 ng/ml respectively.
8. Incubate plate for 48 hours.
9. After about 48 hours alamarBlue (aB) should be added to the plate as follows. Add 10 μl of aB to all of the drug screening wells, the Tryps only wells, the Dimin. control wells and the Media+aB wells (DO NOT add the aB to the Drug+Media blank wells or the Media only wells).
10. Return to the incubator for a further 72 hrs.
11. Once a total of 72 hours has elapsed, read the plate on a Biorad microplate reader at 570 nm and 630 nm.

The data is used to calculate the % Inhibition for each drug and the Diminazene control. Those drugs giving over 50% inhibition go on to have a dose response curve assay.

### B. Dose Response Curves Against Trypanosomes.

As with screening, dose response curves against Trypanosomes are performed in 96 well plates with 3 curves per plate. Initial curves start at 10 μM and decrease to 0.01 μM.
1. Using a haemocytometer count a 10 ml culture of Trypanosomes at optimum growth (ie. high in numbers without any short and stumpy forms apparent). Calculate the dilution factor needed to achieve a concentration of organisms at about 1×10⁵/ml. The Trypanosomes will be diluted a further 1:2 during plate set up, so that the final concentration of organisms should be about 5×10⁴/ml.
2. Drugs are pre-diluted in a 48 well plate. As with the screening plates, a volume sufficient to allow 50 μl to be dispensed to each test and drug blank well is prepared. Dilute Diminazene stock to give 10 ng/ml
3. Add 100 μl of warm media to the Media only and Media+aB wells.
4. Add 50 μl of warm media to the Drug+Media blank wells, Trypanosome only and Diminazene Control wells.
5. Add 50 μl of drug dilution to the appropriate test wells and drug blank wells on the DRC plate.
6. Add 50 μl of Trypanosome suspension to all wells excluding Drug+Media blanks. The addition of Trypanosomes completes the dilution of the drugs. Incubate plate at 37° C. and 5% CO₂ for 48 hours.
7. After about 48 hours alamarBlue (aB) should be added to the plate as follows. Add 10 μl of aB to all of the drug screening wells, the Trypanosome only wells, the Diminazene control wells and the Media+aB wells (DO NOT add the aB to the Drug+Media blank wells or the Media only wells).
8. Return to the incubator for a further 24 hrs.
9. Once a total of 72 hours has elapsed, read the plate on a Biorad microplate reader at 570 nm and 630 nm.

### Results

The results are set out in FIGS. 3A and 3B.

### Example 11

### Screening Against Other Parasites

The results of screens against other parasites are also set forth in Tables 3A, 3B, 4A and 4B.

1. A compound of Formula A: 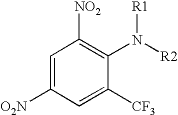
wherein R¹ is C₁-C₅ alkyl, C₃-C₆ branched alkyl, C₄-C₇ cycloalkyl, C₈-C₁₂ fused or bridged polycycloalkyl, or heterocyclic ring, where any of the preceding alkyl, cycloalkyl or heterocyclic ring groups may be singly or multiply substituted with X;
R² is H or R¹; and
X is halo, carbonyl, carboxylic acid, carboxylic ester, carboxamide, substituted carboxamide, hydroxy, alkoxy, thioalkyl, sulphoxide, sulphone, sulphonamide, substituted sulphonamide, phenoxy, substituted phenoxy, phenyl, substituted phenyl, amino, substituted amino (including quaternary ammonium salts), N-oxide, imino, 5-7 membered heterocycle, or substituted heterocycle
said compound optionally being in the form of a pharmaceutically acceptable salt.

2. The compound of claim 1 wherein R¹ is a substituent selected from the substituents listed in FIGS. 2A-2E.

3. The compound of claim 1 wherein R¹ and R² are connected by a bond to form a heterocyclic ring.

4. The compound of claim 3 wherein the heterocyclic ring contains one or more heteroatoms.

5. The compound of claim 1 wherein the compounds contain at least one ring system in addition to the 2,4-dinitro-6-(trifluoromethyl)analine ring.

6. The compound of claim 1 wherein the compounds contain at least one heteroatom in addition to the 2,4-dinitro-6-(trifluoromethyl)analine ring.

7. The compound of claim 1 wherein N(R¹)R² is not pyrrolidino.

8. The compound of claim 1 selected from the group comprising the compounds listed in FIGS. 3A to 3C.

9. The compound of claim 1 selected from the group consisting of:
1-(4-methyl-1-piperazinyl)-2,4-dinitro-6-(trifluoromethyl)benzene;
1-morpholino-2,4-dinitro-6-(trifluoromethyl)benzene;
N-cyclopentyl-2,4-dinitro-6-(trifluoromethyl)aniline; and
N-cyclopentyl-N-methyl-2,4-dinitro-6-(trifluoromethyl)aniline.

10. (canceled)

11. A method for preparing a compound according to claim 1, the method comprising the step of reacting 2-chloro-3,5-dinitrobenzotrifluoride with the corresponding HN(R¹)R².

12. The method of claim 11 wherein the reaction is carried out in the presence of a base.

13. The method of claim 11 wherein the HN(R¹)R² is basic and the reaction is carried out in the presence of an excess of the HN(R¹)R².

14. A composition of claim 1 further comprising a pharmaceutically acceptable carrier or diluent, wherein said composition comprises a comprising a therapeutically-effective amount of a compound of Formula A.

15. The composition of claim 14 that is adapted for topical, oral, parenteral, or aerosol delivery.

16. The composition of claim 14 in a biocompatible, biodegradable matrix, for delivery as an implant.

17. A method of treating a parasitic infection in a subject comprising the step of administering to the subject an effective amount of a compound according to claim 1.

18. The method of claim 17 wherein the parasitic infection is caused by an endoparasite or an ectoparasite.

19. The method of claim 17 wherein the parasitic infection is caused by a parasite selected from the group consisting of: trypanosomes; haemoprotozoa and parasites capable of causing malaria; enteric and systemic cestodes including taeniid cestodes; enteric coccidians; enteric flagellate protozoa; filarial nematodes; gastrointestinal and systemic nematodes and hookworms.

20. The method of claim 19 wherein the trypanosomes are selected from the genera Trypanosoma and Leishmania; the haemoprotazoa are selected from the genera Plasmodium; the taeniid cestodes are selected from the genera Echinococcus; the enteric coccidians are selected from the genera Eimeria and Cryptosporidium; the enteric flagellate protozoa are selected from the genera Giardia; the gastrointestinal and systemic nematodes and hookworms are selected from the genera Amidostomum, Trichostrongylus, Tenorastrongylus, Nippostrongylus, Heligmonina, Boreostrongylus, Ancylostoma; and the filarial nematodes are selected from the genera Wucherieria, Onchocera and Dirofiiaria.

21. The method of claim 17 wherein the parasitic infection is caused by a parasite selected from the group consisting of: Cryptosporidium andersoni, Cryptosporidium parvum, Cryptosporidium muris, Cryptosporidium hominis, Cryptosporidium wrairi, Cryptosporidium felis, Cryptosporidium canis, Cryptosporidium baileyi, Cryptosporidium meleagridis, Cryptosporidium galli, Cryptosporidium serpentis, Cryptosporidium saurophilum and Cryptosporidium molnari; Echinococcus granulosus, E. multilocularis, E. vogeli, E. oligarthrus; Trypanosome rhodesiense, T brucei, T cruzi; G. intestinalis; L. brasiliensis, L. donavani, L. ethiopica L. mexicana L. peruviana, L. tropica, L. major; L. infantum, Plasmodium falciparum, P. humain et simian; Caenorhabditis elegans, Caenorhabditis briggsae, Caenorhabditis drosophilae, Caenorhabditis japonica, Caenorhabditis maupasi, Caenorhabditis plicata, Caenorhabditis remanei, Caenorhabditis sonorae, Caenorhabditis sp. CB5161, Caenorhabditis sp. DF5070, Caenorhabditis sp. PS1010, Caenorhabditis sp. SB341 Caenorhabditis vulgaris, Amidostomum fulicae, A. acutum, Trichostrongylus colubriformis Tenorastrongylus josephi, Nippostrongylus brasiliensis, Nippostrongylus witenbergi, Heligmonina nevoi; Boreostrongylus seurati, Boreostrongylus minutes, Heligmosomoides polygyrus, Wuchereria bancrofti, Onchocerca volvulus, Dirofilaria immitis, Schistosoma mansoni, S. haematobium, S. japonicum, Blastocytis hominis, Pediculus humanis capitis, Onchocera volvulus, Sarcoptes scabei, Trichomonas vaginalis, Toxocaria canis, T. cati and Toxoplasma gondii.

22. The method of claim 17 wherein the parasitic infection is caused by a parasite selected from the group consisting of: Caenorhabditis elegans, Trypanosoma rhodesiense, T. brucei, T. cruzi, Leishmania donovani, Plasmodium falciparum, Cryptosporidium, Giardia, or Echinococcus multilocularis.

23. A method of treating a parasitic disease in a subject comprising the step of administering to the subject an effective amount of a compound according to claim 1, wherein said disease is selected from the group comprising: trypanosomiasis, malaria, coccidiosis, leishmaniasis, giardiasis, hookworm infection, Chagas disease, Schistosomiasis (bilharzia), Blastocystosis, cryptosporidiosis, filariasis, head, pubic and body lice infection, ascariasis, onchocerciasis (River blindness), scabies, toxocariasis and toxoplasmosis.

24. The method according to claim 17 wherein the compound is administered by a route selected from the group comprising: oral administration, administration by inhalation, topical administration, intramuscular administration or intravenous administration.

25. A method of protecting a subject against a parasite infection comprising the step of administering to the subject a prophylactically effective amount of a compound according to claim 1.

26-36. (canceled)

37. The method according to claim 23 wherein the compound is administered by a route selected from the group comprising: oral administration, administration by inhalation, topical administration, intramuscular administration or intravenous administration.
